(19) United States
(12) Patent Application Publication
(10) Pub. No.: US 2023/0157703 A1
(43) Pub. Date: May 25, 2023

(54) SYSTEMS AND METHOD FOR FORMING BIPLANAR OSTEOTOMIES

(71) Applicants: Smith & Nephew, Inc., Memphis, TN (US); Smith & Nephew Orthopaedics AG, ZUG (CH); Smith & Nephew Asia Pacific Pte Limited, Singapore (SG)

(72) Inventors: Paul Alexander Torrie, Marblehead, MA (US); Dennis Colleran, North Attleboro, MA (US); Miles Malone, New Durham, NH (US); John Albert Slusarz, Jr., Hopedale, MA (US); Jason Hamilton, Dartmouth, MA (US); Jacob Mercer, Quincy, MA (US); Rebecca Holmberg, Billerica, MA (US)

(21) Appl. No.: 17/795,567

(22) PCT Filed: Jan. 29, 2021
(86) PCT No.: PCT/US2021/015715

§ 371 Date: Jul. 27, 2022

Related U.S. Application Data
(60) Provisional application No. 63/108,238, filed on Oct. 30, 2020.
(60) Provisional application No. 63/054,561, filed on Jul. 21, 2020.
(60) Provisional application No. 62/967,252, filed on Jan. 29, 2020.

(51) Int. Cl. A61B 17/15; A61B 17/80; A61B 34/10

(52) U.S. Cl. CPC A61B 17/151; A61B 17/8057; A61B 17/8095; A61B 34/10; A61B 2090/067

(57) ABSTRACT

Apparatus and methods are disclosed for determining and placing a first bony segment in relation with a second bony segment, both segments belonging to the same bone, including cutting said bone partially, to separate it into the first and second bony segment. The two bony segments are linked together by a bony hinge and placing the two boney segments in relation to each other may include distracting both bony segments around said hinge. The hinge may be configured to allow distraction of both bony segments around a single degree of freedom of the hinge and accommodate corrections in two substantially orthogonal planes.

### CROSS-REFERENCE TO RELATED APPLICATIONS

This application claims benefit to Provisional Patent Application No. 62/967,252; filed Jan. 29, 2020; and Provisional Patent Application No. 63/054,561; filed Jul. 21, 2020; and Provisional Patent Application No. 63/108,238; filed Oct. 30, 2020, all titled “SYSTEMS AND METHOD FOR FORMING BIPLANAR OSTEOTOMIES”; herein incorporated by reference in their entirety.

This application incorporates by reference commonly owned Patent Application PCT/US20/019094 filed Feb. 20, 2020; and Provisional Patent Application No. 62/808129 file Feb. 20, 2019, both titled “SYSTEM AND METHOD FOR HIGH TIBIAL OSTEOTOMY”.

### FIELD OF THE INVENTION

This invention is related to surgical apparatus and methods in general, and more particularly to apparatus and methods for forming a biplanar osteotomy, to realign bones.

### BACKGROUND

Osteotomies of the lower limbs are an important technique for modifying the loadbearing geometry of the knee. For example a tibial osteotomy may treat knee osteoarthritis by adjusting the geometry of the knee joint to transfer weight bearing loads from arthritic portions of the joint to relatively unaffected portions of the joint. As another example tibial or femoral osteotomies may address abnormal knee geometries, e.g., due to birth defect, injury, etc. Most lower-limb osteotomies are designed to adjust the manner in which the load is transferred across the knee joint. One method of adjusting the orientation of the tibia, is an open wedge technique, represented in FIG. 1B wherein a cut is made into the upper portion of the tibia, forming two segments of the tibia connected by a boney hinge 81. The tibia is then manipulated to separate the two segments and open a wedge-like opening in the bone, and then the bone is secured in this position (e.g., by screwing metal plates to the bone or by inserting a wedge-shaped implant into the opening in the bone), thereby reorienting the lower portion of the tibia relative to the tibial plateau and hence adjusting the manner in which load is transferred from the femur to the tibia. FIG. 1B shows an exemplary open-wedge high tibial osteotomy in association with a knee joint 75 between a femur 70 and a tibia 80. FIG. 1B is an anterior-posterior view. A planar cut 90 at a selected angle β relative to a first reference axis A of the knee joint 75 can be made. This is a partial cut, i.e., not a through cut, and can extend from an external surface boundary 92 at the intersection of the planar cut 90 with the outer surface of the tibia 80 to a second boundary 94 at the selected cutting depth illustrated as distance L in FIG. 1B. The second boundary 94 functions as a hinge axis line (also referenced with numeral 94) for opening a wedge angle γ between first and second opposing faces 96, 98 of the cut 90, as illustrated by arrows C in FIG. 1C. The location of the first and second boundaries 92, 94, the angle β of the planar cut 90 relative to the reference axis A, and the wedge angle γ may be determined during the pre-operative planning stage for correcting a condition of the particular patient. Typically, these are all determined such that the planar cut 90 extends along the coronal plane only, with the second boundary (94) defining an anterior-posterior line substantially parallel to the sagittal plane. Typically, these are all determined according to a single plane correction, in this case a coronal correction angle. However there may be times when the anterior-posterior slope of the tibial plateau may require tilting in the sagittal plane (reference FIG. 1A) for example to further aid in an ACL or PCL condition. This therefore requires two substantially orthogonal corrections to be superimposed upon each other, hereinafter defined as a biplanar correction, represented in FIG. 2A. This may result in a twisting of the remaining boney hinge 81, which may crack 100 (FIGS. 2A and 2B). Referring back to FIG. 1C which shows a single correction plane osteotomy similar to the left hand side of FIG. 2A, in order to accommodate an additional sagittal plane correction, at least one of the first and second opposing faces 96, 98 must now be rotated relative to each other such that opening angle γ varies from anterior to posterior sides. Some attempted solutions have tried adding a stress-relieving hole at the apex or boundary 94 of the cut, to minimize propagation of any cracks, but this has not sufficiently addressed the needs of the procedure and cracking may still occur. Other attempted solutions if a crack is detected, is a more conservative rehab regime. However, this requires additional post-op follow up and care. Therefore, there is a need to provide an improved method and systems for controlling stresses on a boney hinge while correcting alignment of two segments of a bone, accommodating a biplanar correction.

### BRIEF DESCRIPTION OF THE DRAWINGS

For a detailed description of example embodiments, reference will now be made to the accompanying drawings in which:

FIG. 1A shows a body defining planes through the body for reference purposes;

FIG. 1B shows a tibial osteotomy in the coronal plane only, for reference purposes;

FIG. 1C shows a tibial osteotomy distracted in the coronal plane only, for reference purposes;

1D shows a femur osteotomy open and closed wedge, in the coronal plane only, for reference purposes;

FIG. 2A represents a possible result of a biplanar correction with a boney hinge axis or boundary oriented parallel to the sagittal plane, and thereby accommodating a single plane only;

FIG. 2B shows a boney hinge with a crack formed while twisting the opening to accommodate a second orthogonal correction, for reference purposes;

FIGS. 3A and 3B represent X-ray images including an anterior-posterior view (left) and a sagittal view (right) image of a knee joint respectively;

FIG. 4A represents a model used to calculate an adjusted hinge axis, in accordance with the present disclosure;

FIG. 4B graphically represents the varus angle, AP angle and resultant adjusted hinge axis, in accordance with the present disclosure;

FIGS. 4C and 4D schematically represent some example adjusted hinge axis angles of a tibia, in accordance with the present disclosure;

FIGS. 5A and 5B schematically represent some example adjustment hinge axis angles of a tibia, in accordance with the present disclosure;

FIGS. 6A-6C schematically represent an example method and system for performing an open wedge biplanar osteotomy on a tibia, in accordance with the present disclosure;

FIGS. 7A-7F schematically represent an example method and system for performing a closed wedge biplanar osteotomy on a femur, in accordance with the present disclosure;

FIGS. 8A-8E illustrate an aimer assembly and components thereof, for placing a hinge pin at a target modified angle through the bone, in accordance with the present disclosure;

FIG. 9 illustrates an anterior cut guide in accordance with the present disclosure;

FIG. 10A-10F illustrate various views of a bone spreader, in accordance with the present disclosure;

FIG. 11A-11C illustrate various views of alternative bone spreader embodiments, in accordance with the present disclosure;

FIG. 12A-12D illustrate various views of a bone plate having a complex aperture, in accordance with the present disclosure;

FIG. 13A-13C illustrate torques on a boney hinge having a modified hinge axis angle and a resultant preferable plate location, in accordance with the present disclosure;

FIG. 14A-14B illustrate an alternative embodiment of a bone plate having a complex aperture, configured to be placed at a location to reduce torque on the boney hinge, in accordance with the present disclosure;

FIG. 15A-15E illustrate an alternative embodiment of a bone plate configured to reduce torsional moments on the tibia as a result of modifying the hinge axis angle, in accordance with the present disclosure;

FIG. 16A-16D illustrate a system for adjusting compression on a boney hinge in accordance with the present disclosure;

FIG. 17A-17C illustrate a method of compressing a boney hinge using an external compression tool; and

FIG. 18A-18C illustrate a plate and method of ACL repair or reconstruction, concomitant with an open wedge osteotomy of a tibia.

### SUMMARY

Various terms are used to refer to particular system components. Different companies may refer to a component by different names—this document does not intend to distinguish between components that differ in name but not function. In the following discussion and in the claims, the terms “including” and “comprising” are used an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

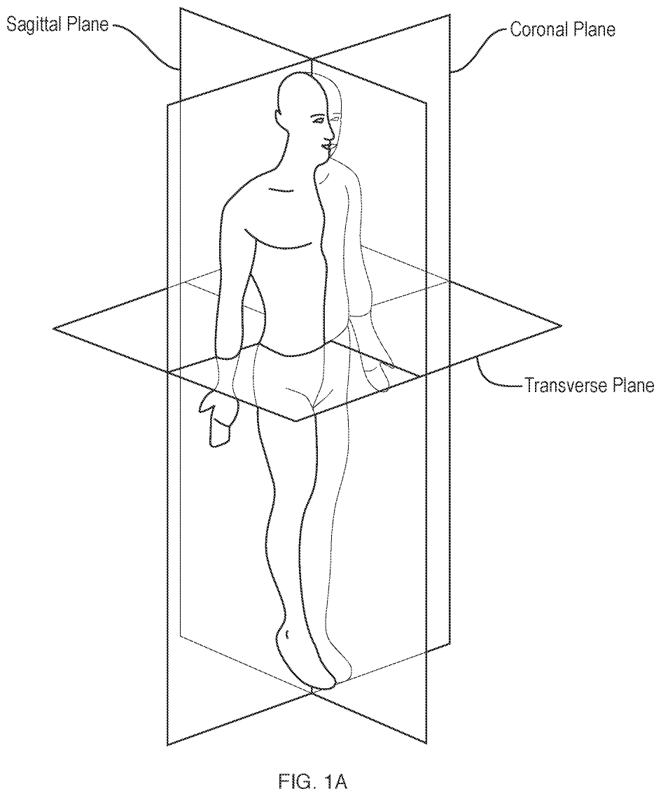

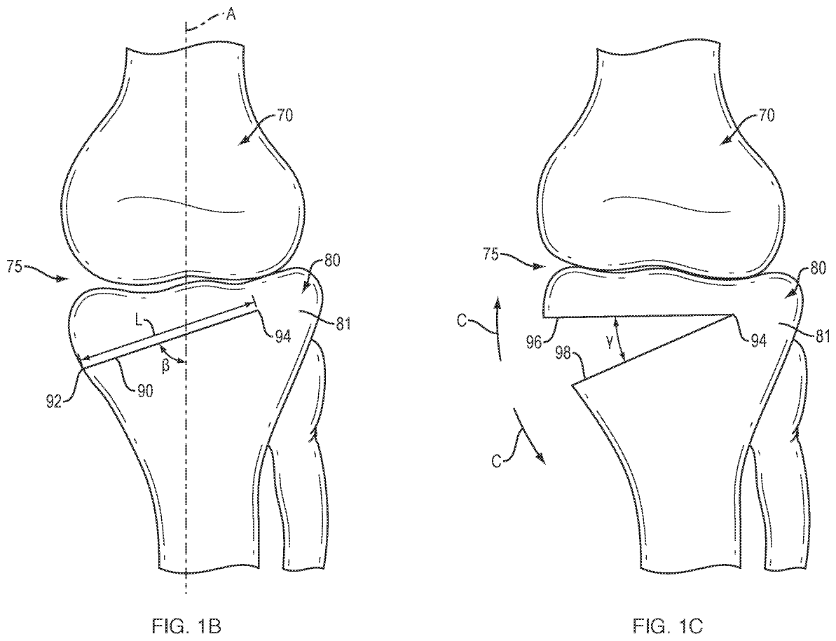

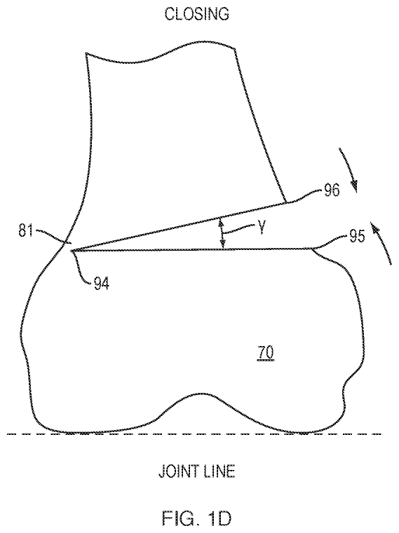

Generally, this disclosure is directed to a realignment of a bone, which may include forming a wedge in the bone that may be an open wedge, tibial osteotomy of the knee, and is intended to provide a cut having a predetermined or calculated apex. This predetermined apex or boundary defines an axis of rotation while opening the osteotomy, the axis determined to incorporate both coronal and sagittal corrections. While existing methods include forming an osteotomy 90 accounting for a single plane correction only, as discussed with reference to FIG. 1B-1D, this disclosure modifies the boney hinge boundary or axis to include a rotation angle θ about a longitudinal axis of the bone, A-A that may be calculated or determined to accommodate for the two orthogonal planes of correction superimposed on each other, hereinafter termed “biplanar correction” or “biplanar osteotomy”. The two orthogonal planes include non-zero corrections in both the sagittal and coronal planes. This rotation angle θ may define a resultant hinge axis angle or orientation; hereinafter termed “modified hinge axis” angle. Compared to a method wherein an osteotomy is formed accounting for a single plane correction only such as the coronal plane only, and then the osteotomy is twisted to accommodate the second plane correction, this method avoids or reduces the need for twisting of the bony hinge. This may mitigate weakening, creating concentrated stresses on, or cracking the bone hinge. A biplanar correction may include forming a single plane osteotomy, the single plane oriented at an angle that accommodates two orthogonal planar corrections. The present invention may also be directed towards a closed wedge tibial osteotomy, utilizing similar principals. For example, in FIG. 1D, the present invention may be implemented for an osteotomy of a femur 70. The present invention may be implemented for a closed wedge osteotomy of any bone. Shown in FIG. 1D, the bone may be removed as a wedge between boundary 94, 95 and 96 and the bone rotated (see arrows) to a closed or abutting position. The present invention determines an improved orientation of the boundary. The present invention may be implemented for any osteotomy on any bone where two superimposed substantially orthogonal correction angles that are non-zero are desired.

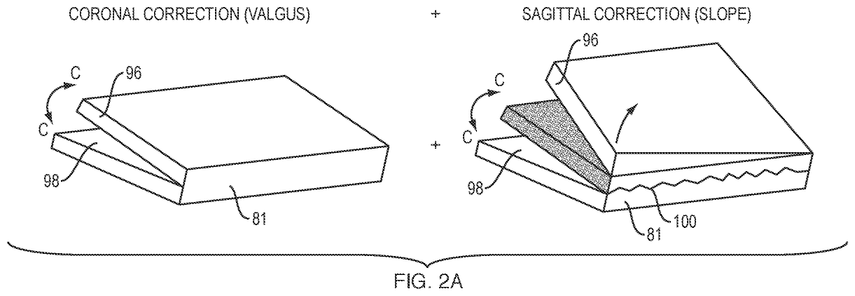

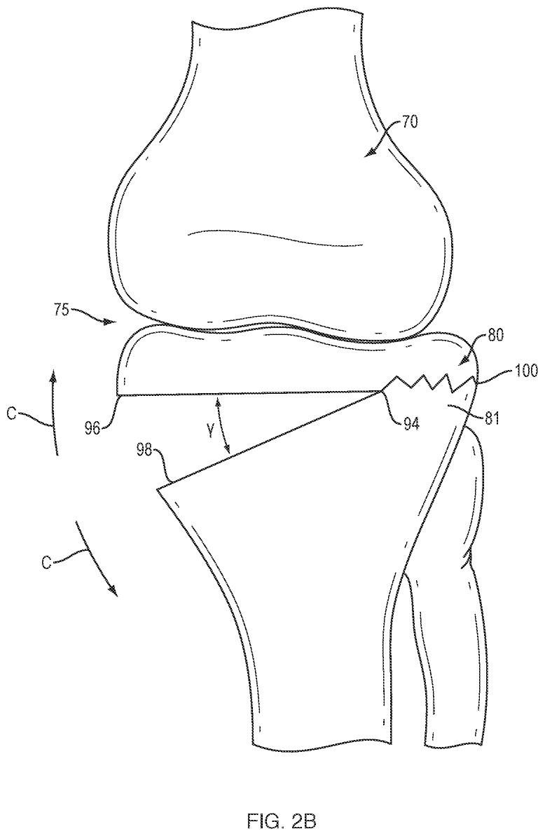

Various embodiments are directed to a surgical method of attachment of a first bony segment in relation with a second bony segment, both segments belonging to a same bone. The method may include cutting the bone partially, to separate it in two bony segments linked together by a bony hinge and distracting or rotating both bony segments around the bony hinge, wherein the bony hinge is configured to rotate both bony segments around a single degree of freedom of the bony hinge and accommodate two substantially orthogonal correction planes. Twisting the bony hinge as shown in FIG. 2A distracts or rotates the bony segments in at least two degrees of freedom. In some embodiments, the method may include preoperative steps of determining the relative three-dimensional positions of both bony segments to obtain the final desired alignment of the two bony segments, the relative three-dimensional position including the two substantially orthogonal correction planes and determining the orientation and position of the bony hinge. In some embodiments the two substantially orthogonal planes include the sagittal plane and the coronal plane. In some embodiments determining the orientation and position of the hinge, comprises using a look-up table. In some embodiments determining the orientation and position of the hinge, comprises using a computer program. In other embodiments determining the orientation and position of the hinge, comprises using a mechanical computer, wherein two angles may be input into a mechanical construct that mechanically resolves the rotation and wedge angle via cams and linkages. In some embodiments after determining the orientation and position of the bony hinge, the method may include drilling a hole through the bone to form a hinge axis at the determined orientation and position. In some embodiments, determining the orientation and position of the bony hinge comprises determining a rotation angle (0) of the hinge axis relative to a vertical plane, or sagittal plane of the bone, about a longitudinal axis of the bone. The method may include the preoperative steps of determining the relative three-dimensional positions of both bony segments to obtain the final desired alignment of the two bony segments, the relative three-dimensional position including the two substantially orthogonal correction planes and determining a final wedge opening angle. The method may include determining the final wedge-opening angle using either a look-up table or a computer program. The method may include rotating the two bony segments to the final wedge-opening angle.

An alternative surgical method is disclosed for attaching a first bony segment in relation with a second bony segment, both segments belonging to a same bone. The method includes cutting partially into the bone, cutting being implemented until obtaining a partial cut, which separates partially the bone into two bony segments linked together by a bony hinge, the bony hinge defining a hinge axis that is oriented at a non-zero angle relative to a sagittal plane through the bone. The method also includes rotating the first bony segment with respect to the second bony segment about said bony hinge about a single degree of freedom until obtaining a desired three-dimensional alignment of the two bony segments, including a biplanar correction as defined herein, and reaching a position in which facing sides of said bony segments are separated from each other by a predetermined opening angle. In some embodiments, the method may also include determining a hinge rotation angle relative to the sagittal plane using a targeted varus correction angle input and a targeted anterior-posterior slope correction angle input, thus defining a hinge axis angle that provides rotation of the first bony segment with respect to the second bony segment around the single degree of freedom. In some embodiments, the method may include determining the hinge rotation angle (θ) using physical charts. In some embodiments, the method may include determining the hinge rotation angle (θ) using a computer program. In some embodiments, the method may include drilling a hole and thus creating a boney hinge axis. In some embodiments, the method may include drilling the hole using preplanning navigation. In some embodiments, the hole may be drilled using a computer piloted robot whose working head is able to be moved according to at least three degrees of freedom. In some embodiments, the method may include cutting the bone, implemented by using a computer piloted robot whose working head is able to be moved according to at least three degrees of freedom, the working head supporting a cutting tool. In some embodiments, the method may include using a cutting guide to guide the orientation of the cutting tool.

A further method is disclosed of controlling a computer piloted robot having a working head that moveable according to at least three degrees of freedom, in order to implement a surgical method of attachment of a first bony segment in relation with a second bony segment, both segments belonging to a same bone. When a cutting tool is coupled to the working head, this method includes causing the cutting tool to cut partially the bone until obtaining a partial cut, which separates partially the bone in two bony segments linked together by a bony hinge, the bony hinge defining a single hinge axis. The method also includes rotating the two bony segments about a single degree of freedom to reorient the first bony segment relative to the second bony segment to a desired orientation including non-zero corrections in two substantially orthogonal planes. In some methods, the two substantially orthogonal planes may include the coronal and sagittal correction. In some methods the computer controlled robot may be in communication with a computer, the computer configured to preoperatively or intra-operatively determine the position and direction of the partial cut, calculating its depth, calculating an opening angle and the relative position of the first bony segment with respect to said second bony segment necessary to obtain the final desired alignment of the two bony segments, the final desired alignment including a biplanar correction.

A non-transitory computer-readable medium is also disclosed that stores a program that, when executed by a processor is configured to cause the processor to receive a value indicative of a first correction angle of a bone and receive a value indicative of a second correction angle of a bone, substantially orthogonal to the first correction angle. This processor is configured to determine a value indicative of a modified bony hinge axis angle including both the first and second correction angle. This processor may also be configured to determine a value indicative of a final wedge opening angle of a first bony segment of the bone relative to a second bony segment of the bone accommodating both the first and second correction angle. In some embodiments the processor may be communicably coupled to a computer piloted robot having a working head that is moveable according to at least three degrees of freedom, and wherein the program, when executed by the processor is configured to cause the computer piloted robot to orient the working head to the determined modified bony hinge angle, determined by the processor. In some embodiments, the computer piloted robot may be in communication with navigation sensors, configured to aid in orienting the working head at the determined modified bony hinge angle. In some embodiments when a cutting tool is coupled to the working head, the processor may be configured to cause the computer piloted robot to cut partially into the bone with the cutting tool until obtaining a partial cut which separates partially the bone into the two bony segments linked together by a bony hinge, the bony hinge oriented at the modified bony hinge axis. In some embodiments when a drilling tool is coupled to the working head, the processor may be configured to cause the computer piloted robot to drill into the bone along the modified bony hinge axis. In some embodiments when a wedge-opening tool is coupled to the working head, the processor may be configured to cause the computer piloted robot to distract the two bony segments to the final wedge opening angle by rotating the first bony segment relative to the second bony segment about a single degree of freedom. In some embodiments when a cutting guide is coupled to the working head, the processor may be configured to cause the computer piloted robot to orient the cutting guide in a desired orientation according to the determined modified boney hinge axis.

In addition, an example surgical method of attachment of a first bony segment in relation with a second bony segment, both segments belonging to a same bone, is disclosed herein including the step of: determining a modified hinge axis angle of a boney hinge that provides a single degree of freedom rotation of the first bony segment relative to the second bony segment, the modified hinge axis angle accounting for a final desired alignment of the two bony segments including two substantially orthogonal plane corrections. An aimer assembly is then placed around the bone. The method may include orienting a guide tube of the aimer assembly in a first orientation and then moving the guide tube along an aimer arm of the aimer assembly to orient an axis of guide tube along the determined modified hinge axis angle. A drill may then be inserted through the guide tube to form a hole through the bone, the axis of the hole defining the modified hinge axis of the bony hinge. In some example methods, moving the guide tube includes sliding the guide tube along the aimer arm. Moving the guide tube may include moving the guide tube up to a target numerical value indicated on the aimer assembly correlating to the modified hinge axis angle. Placing the aimer assembly may include first placing a posterior retractor around a posterior portion of the bone and coupling the aimer arm within a slot of the posterior retractor to place the aimer arm around a medial and anterior portion of the bone. Orienting the guide tube in a first orientation may include manipulating a handle extending from the posterior retractor. The first orientation may be defined as an orientation accounting for only one of the two substantially orthogonal plane corrections. The example method may also include removing the drill from the guide tube; and inserting a hinge pin through the guide tube. The example method may include inserting the hinge pin through the drilled hole to positively-engage a portion of a posterior retractor. The example method may include removing the aimer assembly from the posterior retractor, leaving the hinge pin within the hole and coupled to the posterior retractor; and coupling an osteotomy cutting guide to the posterior retractor and hinge pin.

An example embodiments of an aimer assembly is disclosed herein for forming a hole through a bone at a predetermined modified hinge axis angle. The example assembly includes an aimer arm that links to a retractor and extends around an outer portion of the bone. The assembly also includes a guide tube coupled to the aimer arm configured to move along the aimer arm and thereby around the bone and alter the guide tube orientation relative to the bone. The guide tube is configured to receive a means of forming a bone hole therethrough. The guide tube and aimer arm each include indicating markers that cooperate with each other to indicate a value correlating to the guide tube orientation relative to the modified hinge axis angle. In some example embodiments, the guide tube is slidingly coupled to the aimer arm. In some example embodiments, the guide tube is also configured to receive a hinge pin therethough, to place the hinge pin through the bone. In some embodiments, the guide tube aims the hinge pin towards an engagement mechanism of the retractor. The indicating markers may include numerical values on the aimer arm that correlate to the modified hinge axis angle. The numerical values may provide for a modified hinge axis angle up to 31 degrees relative to a reference angle. The reference angle may define an angle accounting for a single correction plane to the bone, the modified hinge angle accounting for a biplanar correction. The guide tube and aimer arm are configured to disconnect from the hinge pin and retractor, leaving the hinge pin and retractor coupled with each other and the hinge pin remaining through the bone hole.

Example embodiments of a laminar spreader are also disclosed herein, for separating two opposing sides on an osteotomy. An example laminar spreader may include a base having a distal end tapered to slip between the two sides of the osteotomy. The laminar spreader may also include a lifter having a proximal free end and pivotally coupled to the base adjacent the base distal end, that rotate relative to the base defining a cavity between the base and lifter. The laminar spreader may also include a wedge construct operably coupled to the base and configured to axially move a wedge of the wedge construct along the cavity to rotate the lifter and define an angle of separation of the two sides of the osteotomy. In some example embodiments, the wedge construct includes a lead screw orientated parallel to a base longitudinal axis and operable coupled to the wedge. Rotation of the lead screw axially moves the wedge along the cavity. The wedge may engage the lifter at a discrete portion of a top surface of the wedge. The wedge top surface may be disposed within the cavity and may be spaced away from the lifter. The wedge may engage the lifter at a location configured to indicate a separation value of the two sides of the osteotomy.

An example method of attaching a first bony segment in relation to a second bony segment, both segments belonging to a same bone is disclosed, including cutting partially into the bone, the cutting being implemented until obtaining a partial cut, which separates partially said bone in two bony segments linked together by a bony hinge. The bony hinge may define a modified hinge axis angle that is oriented to account for a biplanar correction. The method also includes rotating the first bony segment with respect to said second bony segment around the bony hinge about a single degree of freedom until obtaining a desired three-dimensional alignment of the two bony segments, in which facing sides of said bony segments are separated from each other by a predetermined opening angle. The method also includes fixing the desired three dimension alignment of the two boney segments with a bone plate, the bone plate including a bone engaging surface and holes therethrough, the engaging surface contour and the at least one of the holes' orientation at least partially configured to account for the modified hinge axis angle. In some embodiments, the bone plate bone-engaging surface is contoured to match a contour of the preferred location of the first and second segment of the bone that is approximately parallel with the modified hinge axis. In some embodiments, the bone plate bone-engaging surface is contoured to position the plate at a location approximately facing the modified hinge axis. In some example embodiments, at least one of the holes through the plate defines a complex aperture including two overlapping threaded holes that are oriented at an angle to each other. The angle between the two overlapping threaded holes may be at least partially defined by the modified hinge axis. For a larger predetermined modified hinge axis relative to a hinge axis for a single correction plane only, the angle difference between the two overlapping holes axes is reduced. In some example methods, the bone is a bone of the knee and the method may also include forming a tunnel through the bone for reconstructing an ACL and wherein the two overlapping threaded holes are configured to place at least one fixation member therethrough in a direction that avoids the tunnel through the bone. In some example methods, the holes through the plate defines an inferior plurality of holes configured to orient a fixation member extending therethough at a first angle and a superior plurality of holes configured to orient a fixation member extending therethough at a different angle to the first angle, defining an offset angle configured to account for torsion along the bone that results from the modified hinge axis. The larger the modified hinge axis relative to a hinge axis for a single correction plane only, the larger the offset angle.

Another example plate is disclosed herein for fixing a first boney segment relative to a second segment of the same bone, after the two segments have been distracted around a bony hinge. The bony hinge may be configured to rotate about a single degree of freedom and may be oriented at a modified hinge angle axis to obtain a desired three-dimensional alignment of the two bony segments accounting for two orthogonal correction planes, in which facing sides of said bony segments are separated from each other by a predetermined opening angle. The plate may include a bone-engaging surface configured to match a surface of the plate at a preferred location accounting for the modified hinge angle axis. In some examples, the bone is a bone of the knee and a tunnel may be formed for reconstructing or repairing an ACL. The plate may include an aperture through a superior portion of the plate configured to aim a fixation member to accommodate a concomitant ACL repair or reconstruction. The fixation member may extend through the aperture and adjacent to, but not intersect the tunnel formed therethrough. In some example embodiments, the aperture defines a complex aperture including two overlapping threaded holes that are oriented at an angle to each other and wherein the angle is at least partially defined by the modified hinge axis angle. For a larger predetermined hinge axis angle relative to a hinge axis for a single correction plane only, the smaller the angle is between the two overlapping holes. The two overlapping threaded holes are configured to place at least one fixation member therethrough in a direction that avoids the tunnel for reconstructing the ACL. In some example embodiments, the plate bone-engaging surface is contoured to match surface contours of the bone at the preferred location, which is traverse the distraction and approximately faces the modified hinge axis angle. The plate may include a plurality of apertures through the plate defining an inferior plurality of apertures configured to orient a fixation member extending therethough at a first angle and a superior plurality of apertures to orient a fixation member extending therethough at an offset angle to the first angle, configured to account for torsion along the bone due to the modified hinge axis angle, and wherein the larger the modified hinge axis angle relative to a hinge axis angle for a single correction plane only, the larger the offset angle.

In addition a bone plate flexing system for compression of a bony lateral hinge of an open wedge osteotomy is disclosed, the flexing system for use with a bone plate and fasteners. The bone plate defines a plurality of threaded holes for receiving a plurality of the fasteners therethrough in engagement with the threaded holes. The bone plate includes a bone contacting first surface, an opposing second outer surface, and a thickness extending in a dimension between said the first and second surfaces. The system includes a first shaft having a handle end and a threaded end for threadingly engaging with a first threaded hole of the plurality of the threaded holes. The system also includes a second shaft having a handle end and a threaded end for threadingly engaging with a second threaded hole of the plurality of the threaded holes. The first and second shafts each define a longitudinal axis. The system also includes an apparatus having a distal end configured to engage both shaft handle ends. The apparatus also includes actuation means for moving the first and second shaft handle ends relative to each other while engaged with the bone plate, and thereby elastically flexing the bone plate.

In some example embodiments, the apparatus includes a means of maintaining the bone plate in an elastically flexed configuration. The first and second shafts may both define drill guides. The first and second shaft threaded ends may define a length longer than the plate thickness such that the threaded ends extend beyond the first surface of the plate and form a local standoff between the first surface and bone. The first and second shaft ends may be independently engageable with the bone plate and may by engaged and disengaged by hand. The first and second shaft ends may both engage holes of the bone plate that are disposed either side of a third hole of the plurality of holes. The first and second shaft ends may both engage holes of the bone plate that are disposed at opposing ends of the plate, such as the inferior end and superior end. The first and second shaft ends may both engage holes of the bone plate that are one each side of the osteotomy distraction.

A method of elastically flexing a bone plate and thereby compressing a bony hinge of an open wedge osteotomy is also disclosed herein. The bone plate stabilizes the open wedge osteotomy and includes a superior portion for fixing with a superior side of the osteotomy and an inferior portion for fixing with an inferior side of the osteotomy. The method includes placing a bone plate adjacent an osteotomy of a bone, the bone plate having a first plurality of threaded holes disposed through the plate superior portion and a second plurality of threaded holes disposed though the plate inferior portion. The superior portion of the plate is fixed to the bone. A first elongate body is engaged with one of the holes of the first plurality of holes, and a second elongate body is engaged with one of the second plurality of holes. A plate-flexing tool is then engaged with the first and second elongate bodies. Using the plate-flexing tool, a force is applied via the first and second elongate bodies, to elastically-flex the plate along the plate longitudinal axis. While applying the force, the bone plate is further fixed to the bone, at a location spaced between the first and second elongate bodies, and thereby compressing the bony hinge. In some example methods, applying the force to elastically-flex the plate moves the inferior portion of the plate further away from the bone. The method may include releasing the plate-flexing tool from the first and second elongate bodies after fixing the portion of the bone plate to bone at the location spaced between the first and second bodies, and thereby relaxing the inferior portion of the plate towards the bone.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The disclosure may generally include improved methods and systems to determine, form and fixate a bone alignment adjustment that includes an osteotomy and accommodates a bone alignment with correction in two substantially orthogonal planes. This disclosure may include improved systems and methods of ACL repair or reconstruction that includes a concomitant bone alignment adjustment. These methods and systems may determine a modified open or closed wedge angle and boney hinge axis that accommodates a targeted bone alignment including two substantially orthogonal planes. These two substantially orthogonal planes may be the sagittal and coronal planes of the body. This disclosure may also include methods including calculating or determining an improved hinge-opening angle and hinge axis to accommodate a targeted bone alignment around two substantially orthogonal planes. This hinge-opening angle and hinge axis are configured to rotate the osteotomy about a single degree of freedom only, and thereby require no twisting of the boney hinge to accommodate the biplanar alignment. This disclosure also includes systems for forming and maintaining this improved osteotomy cut angle and/hinge line and hinge opening angle, accommodating for changing forces and torsions on the bone. The osteotomy may be part of a high tibial osteotomy, or a lower (or distal) femur osteotomy. The osteotomy may be part of shoulder-elbow-wrist (SEW) realignment. The osteotomy may be part of a procedure including an open-wedge osteotomy or closed wedge osteotomy. In a closed wedge osteotomy, a wedge of bone is removed and the two segments rotated towards each other. This disclosure may therefore also include determining parameters associated with the size, angle and location of the wedge of bone to be removed, such that upon removal, rotation of the two segments rotates them around a single degree of freedom and accommodates a biplanar correction.

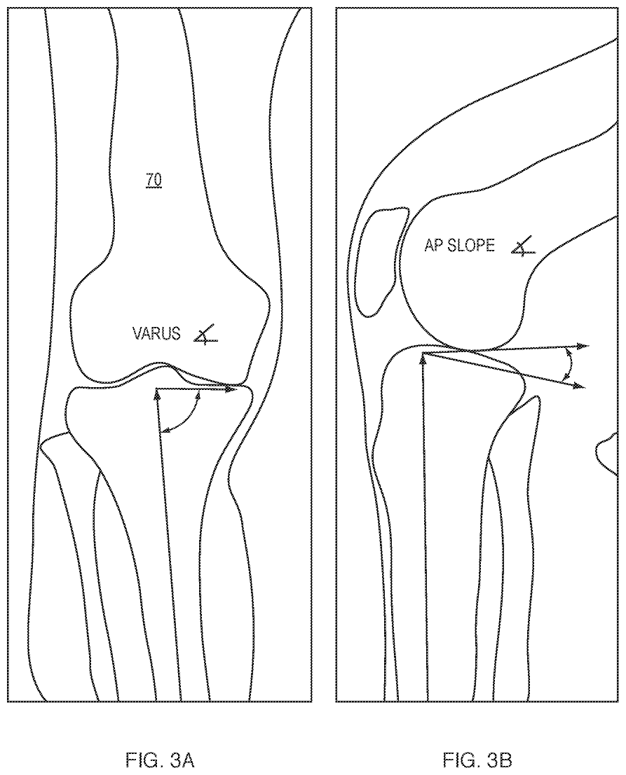

The desired correction angle including the two substantially orthogonal planes may be determined during a pre-operative planning stage of the procedure. For example using a series of several preoperative medical images and data (x-rays echography, MRIs or CAT scans for example), the surgeon knows for example the pathologic angle HKA (Hip-Knee-Ankle) or alternatively SEW (Shoulder, Elbow, Wrist) of his patient. For example, represented in FIGS. 3A and 3B represent two views of the tibia commonly taken by X-ray. FIG. 3A shows an anterior-posterior view, used to determine the varus correction, otherwise defined as a correction in the coronal plane. FIG. 3B shows a sagittal view, used to determine the AP correction otherwise defined as a correction in the sagittal plane. Based on these desired angles, a computer may be configured to determine the desired target alignment of the two bony segments. The computer may include a series of mathematical equations, or a look up table. The computer may include a 3D modelling program that may automatically model the current anatomy orientation based on the medical image data and determine the targeted orientation of the hinge axis. Alternatively or additionally the computer may determine at least one of the starting position (92) on the bone surface, the orientation relative to the sagittal and coronal planes, the depth of the future partial cut, the opening wedge angle of the hinge after distraction or rotation of the bony segments and the relative three dimensional positions of the first bony segment with respect to said second bony segment. The computer may include a 3D modelling program that may interactively model the current anatomy orientation based on medical image data and allow the surgeon to modify a targeted bone alignment and determine at least one of the position, the direction and the depth of the future partial cut, the opening angle of the hinge after rotating of the bony segments and the relative three dimensional positions of the first bony segment with respect to said second bony segment. The computer may be in the form of a tablet, including an “APP” and provide information to the surgeon or surgical technician. The computer may be in communication (directly or wirelessly) with a robot and/or surgical navigation system or 3D localizer configured to place a cutting tool, guide and/or drill in the desired location and orientation, after registering the anatomy with the virtual model.

The computer may also address combined surgeries such as Anterior Cruciate Ligament (ACL) reconstruction with the High Tibial Osteotomies (HTO). In this example, the ACL reconstruction tunnels and osteotomy cut with plating screws could inadvertently intersect, creating negative surgical outcomes. With 3D computer navigation, this issue could be eliminated. Alternatively, rather than using a computer, on the basis of these medical images a chart or look-up table may be referenced directly by the surgeon to determine at least one of the position, the orientation and the depth of the future partial cut, the angle of the hinge axis relative to the sagittal plane and the relative three dimensional positions of the first bony segment with respect to the second bony segment. In other embodiments, a mechanical computer may be used, wherein two angles based on the medical images may be input into a mechanical computer construct that mechanically resolves the rotation and wedge angle via cams and linkages.

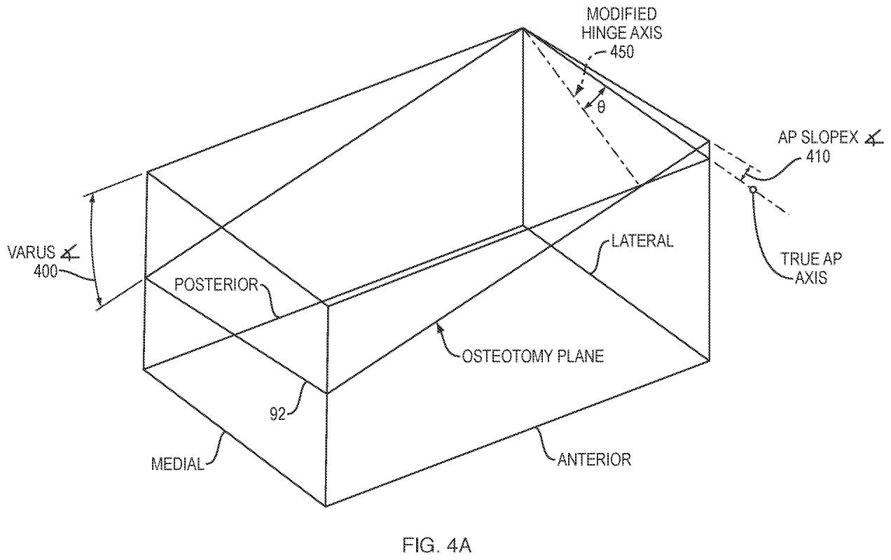

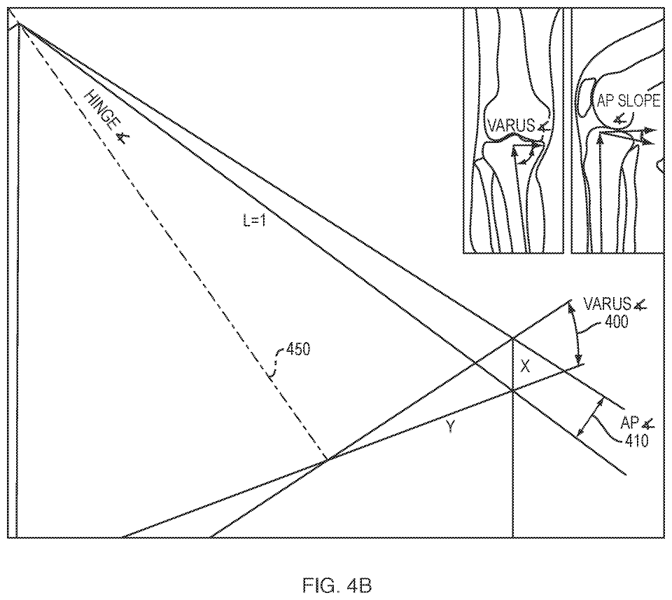

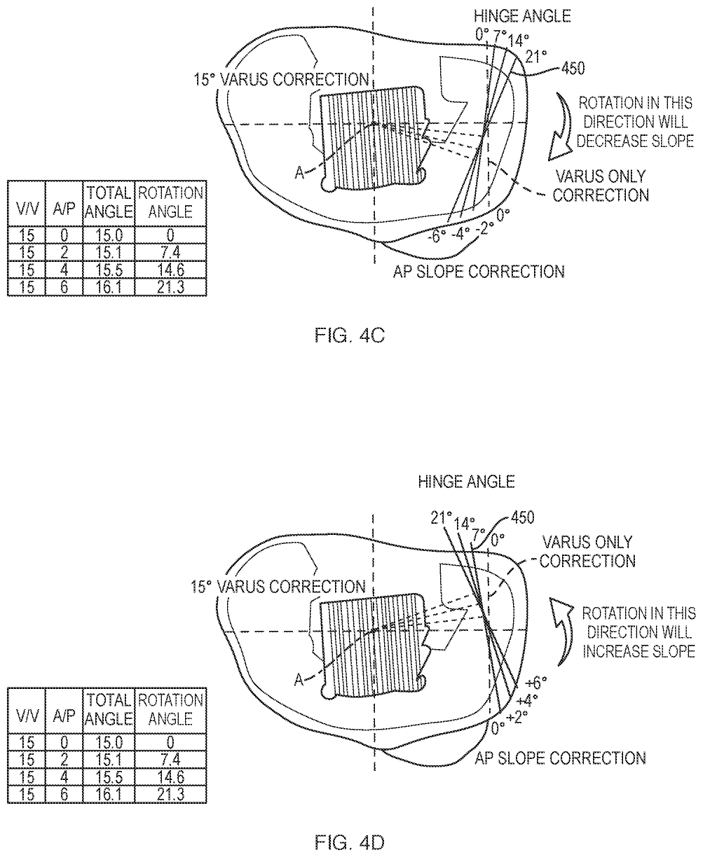

For example for desired Varus and AP slope corrections angles, using for example the two images in FIGS. 3A and 3B, a modified hinge axis orientation may be determined. For a varus correction angle only, the hinge axis typically extends substantially parallel to the sagittal plane and is hereinafter defined as the reference plane. FIG. 4A represents a model of the tibia with the varying input angles. This may be used as a means of calculating the modified hinge axis angle relative to the reference plane that may be incorporated into a computer, or used to form a look up table, as described earlier. Shown in FIG. 4A is an example varus correction angle [symbol] 400, determined using the Anterior-posterior image (FIG. 3A) for example. Shown in FIG. 4A is an example AP slope correction angle [symbol] , 410 determined using the sagittal image (FIG. 3B) for example. For reference purposes, the anterior, posterior, lateral and medial sides are represented and correspond to anterior, posterior and lateral and medial sides of the tibia. For point of reference the true AP Axis may be boundary line 94 in FIGS. 1B and 1C, corresponding to a single plane correction that is parallel to sagittal plane is shown. The equation that may determine the modified hinge axis 450, modified by a rotation angle (θ) relative to the AP axis or relative to the sagittal plane, with known AP≥ 410 and Varus [symbol] 400 using FIG. 4B as reference may look as follows.

$$Solving for X :$$

$$tan ( AP ∢ ) = X / L$$

$$X = tan ( AP ∢ ) \times L ( where L = 1 )$$

$$X = tan ( AP ∢ )$$

$$Solving for Y :$$

$$tan ( Varus ∢ ) = X / Y$$

$$Y = \frac{X}{tan ( Varus ∢ )}$$

$$Solving for Hinge Angle :$$

$$Hinge ∢ = arctan ( \frac{Y}{L} ) = arctan ( Y )$$

$$Hinge ∢ = arctan ( \frac{X}{tan ( Varus ∢ )} )$$

$$Substituting tan ( AP ∢ ) for x :$$

$$Hinge ∢ = arctan ( \frac{tan ( AP ∢ )}{tan ( Varus ∢ )} )$$

Similar calculations may be made for other bones, using similar references. For example, the modified axis may be rotated about a longitudinal axis of a bone and with reference to any plane along the bone that is appropriate, anatomically. For example, the rotation angle θ could be calculated relative to either a sagittal or coronal plane through the bone.

In addition, a new distraction angle γ′ may be determined, and is a resultant angle taking into account the two substantially orthogonal angles; the Varus correction angle [symbol] 400, and the AP slope correction angle [symbol] 410. This new distraction angle γ′ or single combined “Total Angle” in combination with the modified hinge axis angle is configured to orient the first bony segment relative to the second boney segment, with rotation about a single degree of freedom, (without twisting of the two bony segments relative to each other) accounting for at least two substantially orthogonal creation planes.

The equation that may determine the resultant wedge angle γ′ with known AP [symbol] and Varus [symbol] look as follows.

$$Wedge ∢ = \sqrt{{AP∢}^{2} + Varus {∢}^{2}}$$

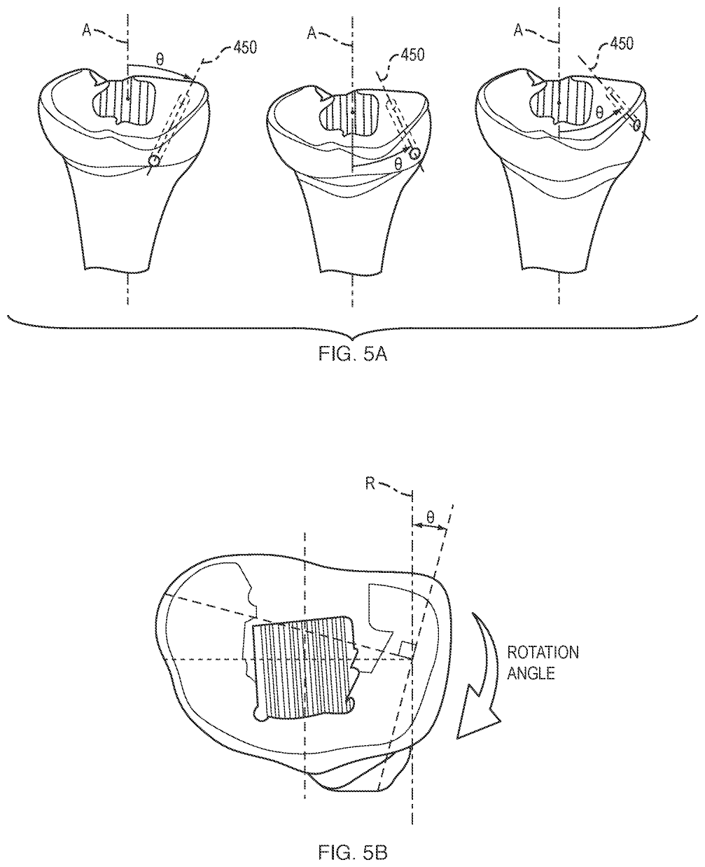

An example look up table, using the above equations is shown below. V/V represents the Varus correction angle [symbol] 400, A/P represents the AP slope correction angle [symbol] 410. “Total Angle” is the resultant Wedge opening [symbol] (γ′), or described above. “Rotation Angle” in the chart below is the “Hinge 4” per the equation above, and is the modification in angular degrees)(° to the hinge line axis, indicated as 0 on FIGS. 5A and 5B. In summary, this algorithm or series of calculations use a plane that is described by angles (lines) in two correction planes: Varus Angle (Coronal Plane) and AP Angle (Sagittal Plane), and resolves that into a “surgically useful” single plane described using an axis in space (Hinge Axis) and single (total) angle.

| | | Total | Rotation |
|---|---|---|---|
| V/V | A/P | Angle | Angle |
| 5 | 0 | 5.0 | 0 |
| 5 | 2 | 5.4 | 21.7 |
| 5 | 4 | 6.4 | 38.6 |
| 5 | 6 | 7.8 | 50.1 |
| 10 | 0 | 10.0 | 0 |
| 10 | 2 | 10.2 | 11.2 |
| 10 | 4 | 10.8 | 21.6 |
| 10 | 6 | 11.6 | 30.7 |
| 15 | 0 | 15.0 | 0 |
| 15 | 2 | 15.1 | 7.4 |
| 15 | 4 | 15.5 | 14.6 |
| 15 | 6 | 16.1 | 21.3 |

FIG. 4C schematically represents an exemplary series of hinge axis angle orientations for a 15 degree Varus correction for a series of AP corrections about the coronal plane in a first direction that reduces the anterior/posterior slope of the tibial plateau. FIG. 4D schematically represents an exemplary series of hinge axis angle orientations for a 15-degree Varus correction for a series of AP corrections about the coronal plane in a second direction that increases the anterior/posterior slope of the tibial plateau. There will be situations where the patient requires an increase in AP slope and instances where the patient needs a decrease in AP slope to optimize a biplanar correction. Improper AP slope can lead to increased risk of ACL rupture. In some cases, ACL reconstruction is completed concurrent with a HTO procedure.

AP slope correction may include increase or decrease the AP Slope for the tibial plateau. The osteotomy will open opposite the boney hinge location. If the hinge is rotated anteriorly (or clockwise) as shown in FIG. 4C the osteotomy will open posteriorly, decreasing the tibial plateau AP slope. If the hinge is rotated posteriorly (FIG. 4D), the osteotomy will open anteriorly increasing the AP slope. In summary, rotating the hinge axis posteriorly will have a positive increase in AP slope. Rotating hinge anteriorly will decrease the AP slope. FIG. 5A shows a view representing three variations of the rotation angle θ of the hinge axis about a longitudinal axis such as axis A-A. In this example, the angle θ is provided relative to the sagittal plane. The left hand image represents rotating the hinge anteriorly, while the right two images represent rotating the hinge axis posteriorly. FIG. 5B shows a top view of a tibia parallel with the transverse plane with an example rotation angle θ (rotation angle). Reference line R-R indicates a typical cut made for a coronal correction only as shown and described in FIGS. 1B and 1C.

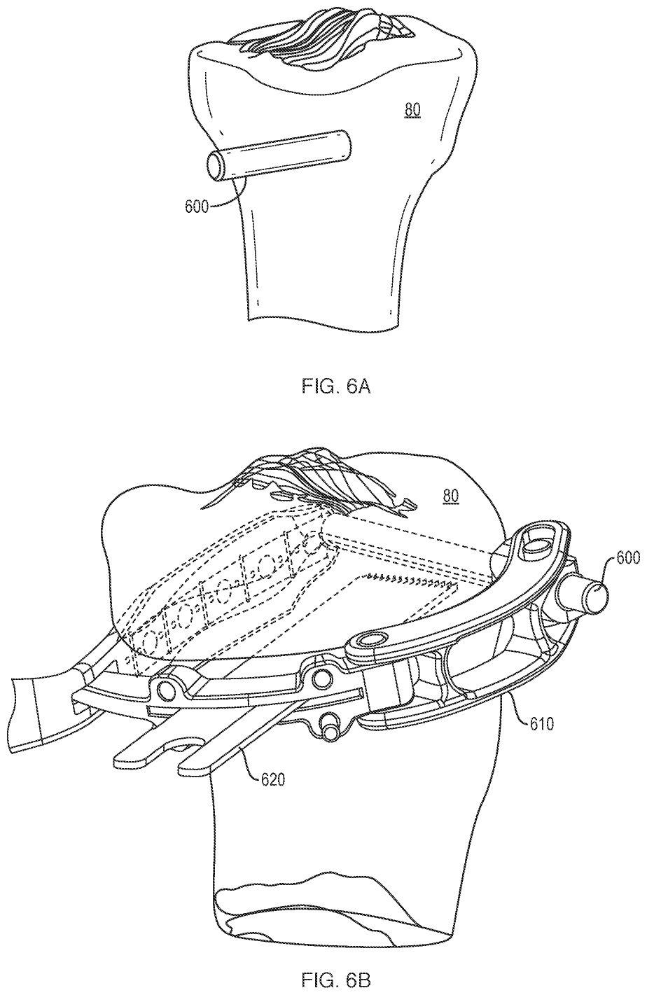

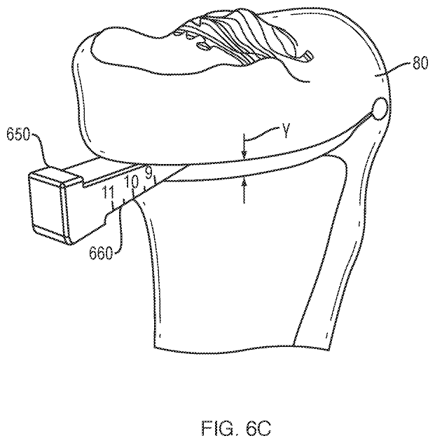

FIG. 6A-6C represent portions of a system and method that may be used to form a biplanar osteotomy through the bone 80 as disclosed herein. The example system and method may include determining the modified hinge axis angle (θ) and location. This may be achieved using a computer program or a look-up table for example. A hinge pin 600 may then be located through the bone at this modified hinge axis location and orientation. A mechanical guide may be used to aid the surgeon in placing this pin 600 along the modified hinge axis angle. For example, a guide construct may include a series of openings or tubes aligned at a series of angles to each other, corresponding with a plurality of discrete modified hinge angles, the openings configured to receive at least one of a guide wire, drill, trephine or pin therethrough. As another example, a guide construct may include a marker or opening slideable between a series of positions, the positions corresponding with a plurality of modified hinge angles, the opening also configured to receive at least one of a guide wire, drill, trephine or pin therethrough.

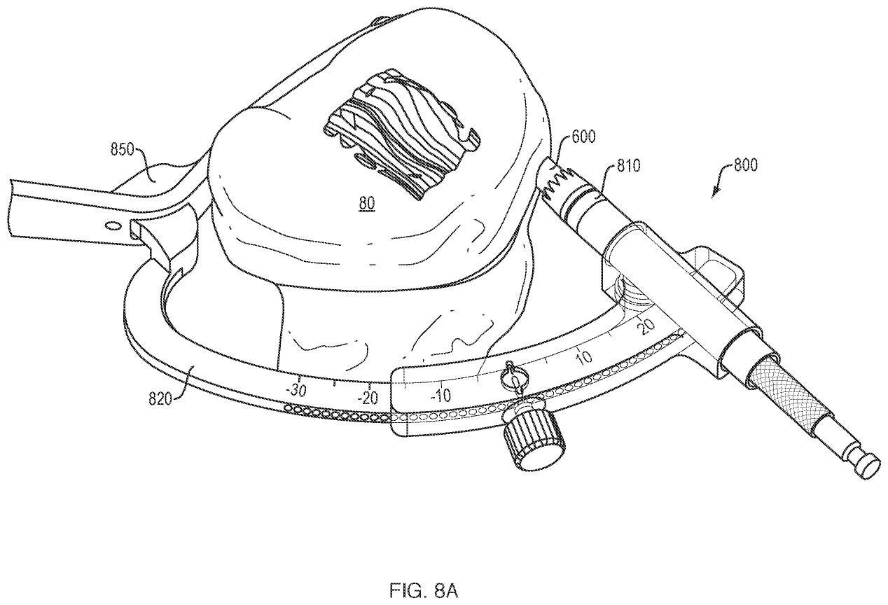

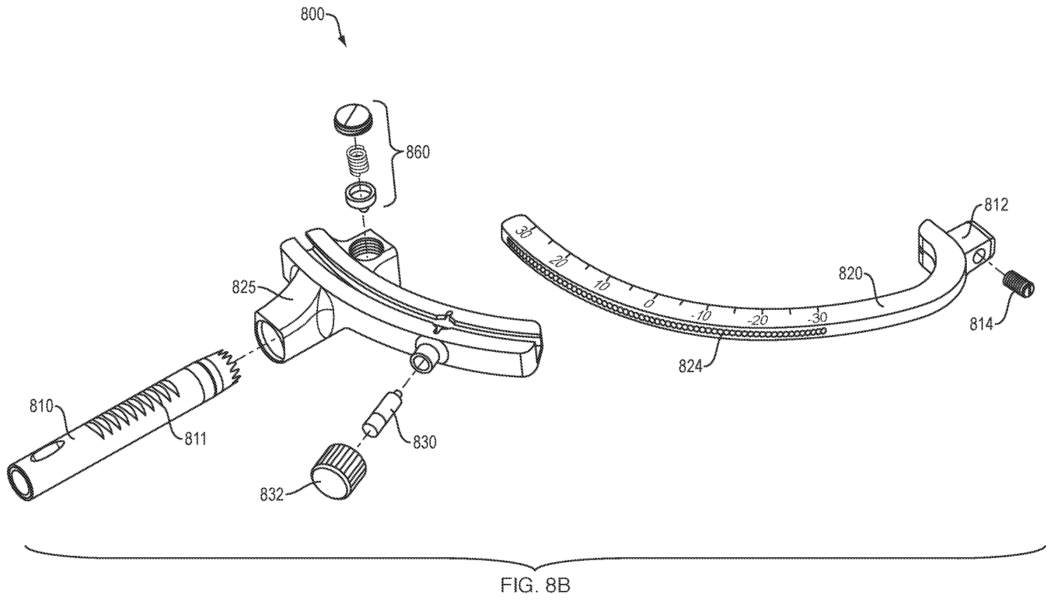

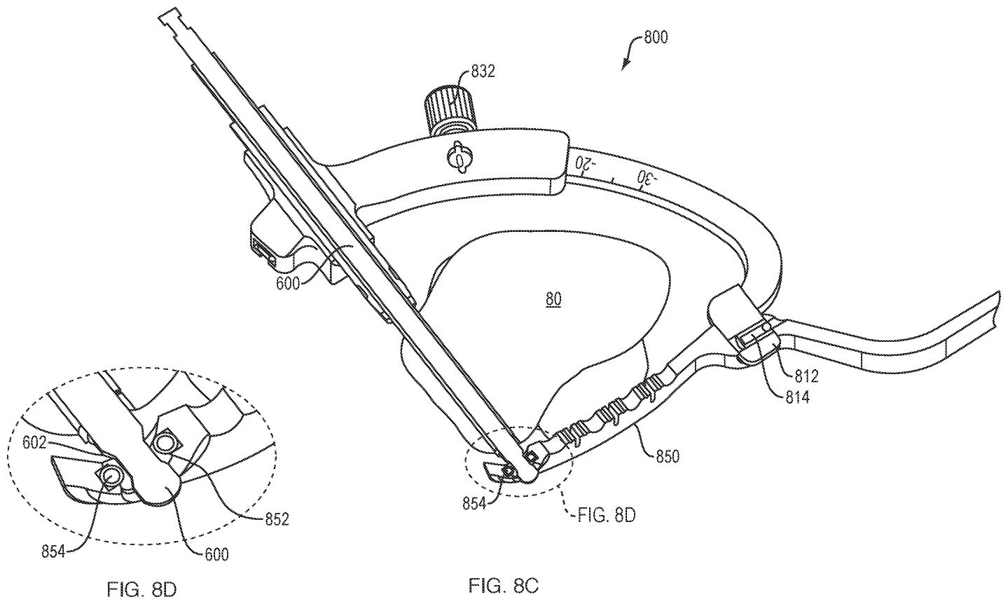

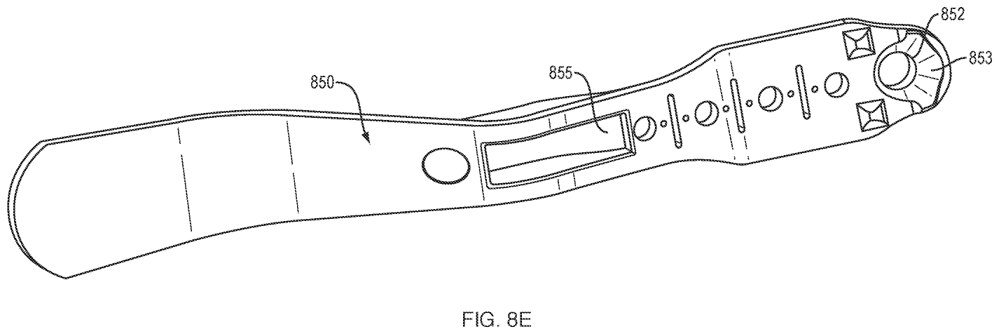

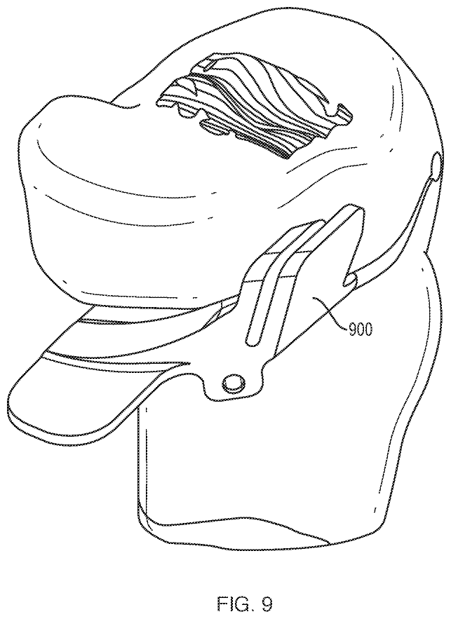

An example aimer assembly 800 is disclosed in at least FIG. 8A-8D. In this embodiment, a drill bit may first be inserted through the opening 810, once the aimer assembly 800 has been aligned, opening 810 aligned with the predetermined modified hinge axis orientation. Drill may be inserted through the bone to create a tunnel corresponding with the hinge line axis at the modified angle θ. Drill may then be removed and a pin 600 put in its stead. Alternatively the drill bit may also double as a hinge pin, and remain in place. A cutting guide 610 may be operationally coupled to the hinge pin 600 to place a cutting tool 620 at a desired location relative to the hinge pin 600. The guide 610 and hinge pin 600 may then be removed and the two bony portions distracted about a single degree of freedom defined by the modified hinge axis angle and thereby achieve the biplanar alignment. This may produce an opening accounting for both the coronal and sagittal correction, with no twisting of the bony hinge, and thereby is a distraction limited to a single degree of freedom.

In alternative systems and methods, a hinge pin 600 may be eliminated and a cutting guide 610 may be configured to place the cutting tool 620 at a desired orientation to form a cut that is adjusted and oriented at an angle to the sagittal plane according a determined modified angle. The terminal end of the cut therefore defines the modified hinge axis 450 or boundary that defines a modified hinge axis, the modified angle (θ) that is non-parallel to the sagittal plane. Alternatively, an aimer assembly 800 may be avoided and a drill bit may be coupled to a working end of a robot operable to orient the drill bit at the desired orientation and location, to orient the hinge pin 600 in the determined orientation and place it into the bone. Alternatively, an aimer assembly 800 may be avoided and a cutting tool may be coupled to a working end of a robot operable to orient the cutting tool at the desired orientation and location, to orient the osteotomy 600 in the determined orientation to form an osteotomy partially through the bone that terminates at a boundary that aligns with the predetermined modified hinge axis.

An alternative system and method may include forming a patient specific cutting guide and/or hinge pin orientation construct. This may be used to place a hinge pin through the bone 80 and/or corresponding cutting plane, accounting for the two substantially orthogonal correction planes. Similar to the previous example disclosed, this example system and method may include determining the modified hinge axis angle and location or a cutting plane orientation and location. This example system and method may include determining a cutting guide surface morphology that conforms to external surfaces contours of the tibia. The guide surface contours may be configured to place the guide in the target location and orientation that forms the predetermined osteotomy. More specifically the guide contours may position the guide and may orient a hinge pin and/or cutting saw 620 through the tibia at the determined location and orientation that realigns the bone with a rotation about a single degree of freedom and accommodates a biplanar correction. The guide morphology may be determined using images (CT or X-ray for example), and 3D images. Determining the 3D surface morphology may include using a computer program. A mechanical guide may be formed or modified according the determined morphology, specific to the patient, based on these images received. For example an inner surface of the guide may be formed or modified to have an inner surface morphology configured to engage and match a determined location of a medial side of the tibial external surface, specific to the patient. Matching is preferably configured to place a saw 620 at the determined starting location and orientation to form the determined osteotomy trajectory. The guide 610 may also include a construct that aligns the hinge pin 600 at the determined angle. The hinge pin alignment construct may include a patient matched reference surface specifically configured to align the hinge pin through the tibia at the determined modified axis angle. The hinge pin patient matched reference surface may cooperate with a surface of the guide or with external surface of the patient bone.

FIG. 6C shows means of forming the determined wedge distraction angle γ′ that accounts for the two substantially orthogonal correction planes. A first means may include includes a wedge 650 with a scale 660. The scale 660 may include values indicative of a wedge opening angle such as angle γ′. Wedge 600 is tapered for gradual insertion and distraction and may be tapped or pushed in up until the determined wedge angle, the scale 660 indicating a distraction angle γ′. To account for both substantially orthogonal correction planes, the wedge may be used to rotate the two bony segments up to the modified distraction angle γ′. Wedge 650 may be selectively coupled to a handle that includes a surface configured to be tapped or hammered on to gradually distract the two boney segments.

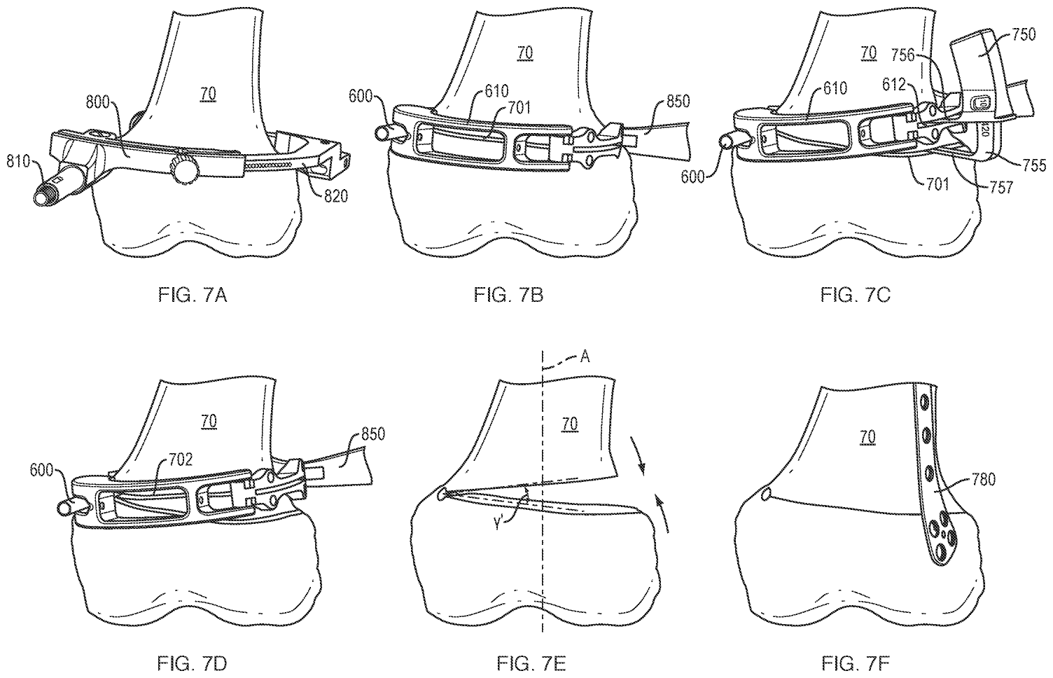

FIG. 7A-7F illustrate an example method of forming a closed wedge biplanar correction on a femur 70. Similar to the previously disclosed method, the example method may begin by determining the modified hinge axis angle and location. This method may also determine a wedge size, and thereby the orientation of two cuts into the bone, as a closed wedge osteotomy removes a wedge portion of bone and the two remaining surfaces rotated towards each other. Predetermining the modified hinge axis and wedge size may be achieved using a computer program, app, or a look-up table for example. An aimer assembly similar to assembly 800 may first engage the femur 70 and the opening 810 may be moved along arm 820 to the determined angle (FIG. 7A). A drill may be inserted through opening 810 to form a hole that aligns with the predetermined modified hinge axis through the femur. Drill may remain in place, to act as the pin or be removed and replaced by a hinge pin 600. The aimer assembly 800 may then be removed, leaving the pin 600 in place and a cutting guide 610 may be operationally coupled to the hinge pin 600 to guide a first cut 701 through the femur 70 up to the hinge pin 600 (FIG. 7B). The cutting guide 610 may then be moved to a second location, while remaining engaged with the hinge pin 600 (FIG. 7C). The cutting guide 610 may be rotated a predetermined amount, to form the predetermined wedge size. A wedge size indicator 755 may be operably coupled to the cutting guide 610 with markings to indicate a wedge size. Indicator 755 may include a first lip 756 that may be inserted through a saw guide slot 612 of the cutting guide 610, and a second lip 757 that may be inserted into the first cut 701. The cutting guide 610 may then be rotated about pin 600 up until the predetermined wedge size as indicated on the indicator 755. The indicator 755 may then be removed and a second cut 702 formed, up to the pin 600. The guide 610 and hinge pin 600 may then be removed and the resultant boney wedge removed to form an opening γ′ having an axis of rotation that accommodated a biplanar correction (FIG. 7E). The two boney portions may then be rotated towards each other about a single degree of freedom defined by the modified hinge axis angle and thereby achieve the biplanar alignment. The two segments may then be fixed in place with a plate 780.

FIG. 8A-8C illustrates details of aimer assembly 800 for placing a hinge pin 600 and thereby orienting a boney hinge axis at the modified hinge axis θ. The assembly 800 as shown may allow up to a thirty degree hinge angle rotation in either direction from reference angle R-R (FIG. 5B). Shown in FIG. 8A, the angle is at zero, and the aimer assembly 800 oriented along reference angle R-R. In use, the surgeon may first place a posterior retractor 850 around a posterior portion of tibia 80, and then link aimer assembly 800 with retractor 850. Aimer 800 may then be adjusted initially to align a guide tube 810 for the varus correction angle only (at zero), and therefore substantially parallel to the sagittal plane, herein defined along the reference angle R-R. Using information from a look up table, computer program or app together with pre-procedure images and planning, the guide tube 810 may then be adjusted by sliding a portion of the aimer 800 around the aimer arm 820 to the determined modified axis angle θ. Guide tube 810 may be configured to receive a drill therethrough and aim the drill through the tibia 80 at the predetermined modified hinge axis angle θ. A hinge pin 600 (as disclosed in FIGS. 6A and 6B) may optionally then be placed within drilled hole at this pre-planned axis angle.

FIG. 8B illustrates an exploded view of the aimer 800. Aimer arm 820 is configured to link to a posterior retractor 850, seen in FIG. 8E, and that may be used to later couple to a construct that guides the osteotomy, as disclosed herein. Aimer arm 820 defines a linking end 812, which may include a ball plunger 814. This may include a biasing member to help maintain the aimer arm 820 within posterior retractor slot. Aimer arm 820 is configured to extend around an outer surface of a patient knee. Aimer arm 820 defines markings such as laser marks, to indicate an angle corresponding to the “Rotation Angle” or modified hinge line axis θ, disclosed herein. Aimer guide 825 may be slidingly coupled to the aimer arm 820. Aimer guide 825 may also include a cannulation for receiving guide tube 810 therethrough such that guide tube 810 may be removed from aimer guide 825. Sliding the guide 825 along the arm 820 and thereby around the bone 80, moves the guide tube 810 around the bone also. Moving the aimer arm 820 to align the marks to a value indicative of a rotation angle θ, is configured to align the guide tube at the determined modified hinge axis angle. Aimer guide 825 may also include means to selectively lock the guide 825 in a target location relative to arm 820. A threaded plunger 830 may extend through arm 825 and may selectively engage a portion of arm 820 to lock the guide 825 at the target modified angle. Knob 832 may operate plunger 830 to selectively lock guide 825. A series of holes or an elongate slot 824 may receive an end of plunger 830, selectively inserted using knob 832. Aimer 800 may also include a construct 860 for retaining tube 810 in position. Construct 860 may include a ratchet plunger, locking cap and spring that may cooperate with ridges 811 on tube 810 to axially fix tube 810 in position within the guide 825.

FIG. 8C shows a cross section through aimer 800 and retractor 850. Ball plunger 814 together with a tapered linking end 812 may insert into a slot 855 of retractor 850. This aligns the aimer arm 820 to the retractor 850, so that the hinge pin 600 will accurately align with retractor 850. Retractor 850 may also be configured to positively engage an end of hinge pin 600. For example, retractor 850 may include an opening 852 with a retaining BalSeal or spring like O-ring 854 to secure the hinge pin 600 to the posterior retractor 850. Opening 850 may include a tapered opening 853, opening towards bone engaging surface of the retractor 850 to guide receipt and alignment of pin 600 with opening 852. The aimer 800 may then be removed leaving the retractor 850 and pin 600 in place and a cut guide may be assembled to the hinge pin 600 and retractor 850. An example cut guide 610 is shown in FIG. 6B that references the hinge pin 600. Cutting guide may be similar in part, with cutting guide disclosed in commonly owned patent application No. 62/808,129 filed Feb. 20, 2019, titled “SYSTEM AND METHOD FOR HIGH TIBIAL OSTEOTOMY”; herein incorporated by reference in its entirety.

The hinge pin 600 may locate the cut guide 610 in the target position and orientation to produce the preferable osteotomy plane angle relative to the modified hinge axis angle θ. The cutting guide 610 may connect to the posterior retractor 850. The hinge pin 600 also provides a barrier or border to the osteotomy, thereby limiting travel of a saw (such as tool 620) creating a high precision osteotomy cut. Cutting guide 610 and pin 600 may then be removed and an anterior cut guide 900 placed adjacent the tibia 80 to orient a transverse cut to relieve the patella tendon. An example anterior cut guide 900 is shown with a lip that may fit between the two boney segments, formed by the osteotomy. Anterior cut guides are disclosed in at least commonly owned patent application No. 62/808,129 filed Feb. 20, 2019, titled “SYSTEM AND METHOD FOR HIGH TIBIAL OSTEOTOMY”; herein incorporated by reference in its entirety. This anterior cut allows the osteotomy opening to occur.

During preplanning, the surgeon may use a series of charts provided, examples of which have been disclosed herein. For example, for a determined Varus correction (degrees) and a determined AP slope Correction (degrees) a modified hinge axis angle orientation (degrees) may be determined and the aimer 800 may orient a guide tube and thereby the hinge pin 600 at this determined axis angle. In addition, with the same two input corrections, a chart may also provide an overall opening angle (Hinge [symbol] ). Alternatively, a wedge opening distance (in mm for example) may be provided in lieu of or in addition to an opening angle γ. In alternative embodiments a computer program or app may convert the two input correction angles and provide the surgeon with the modified hinge angle axis and wedge opening dimension.

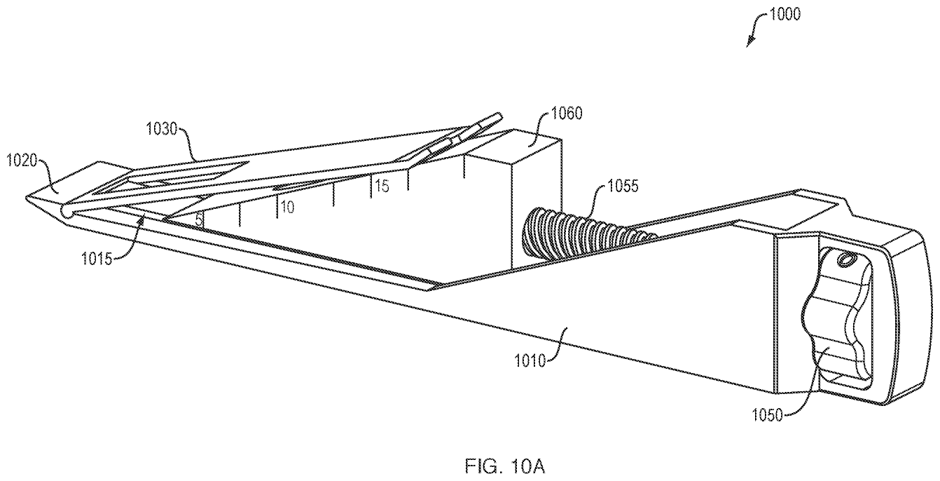

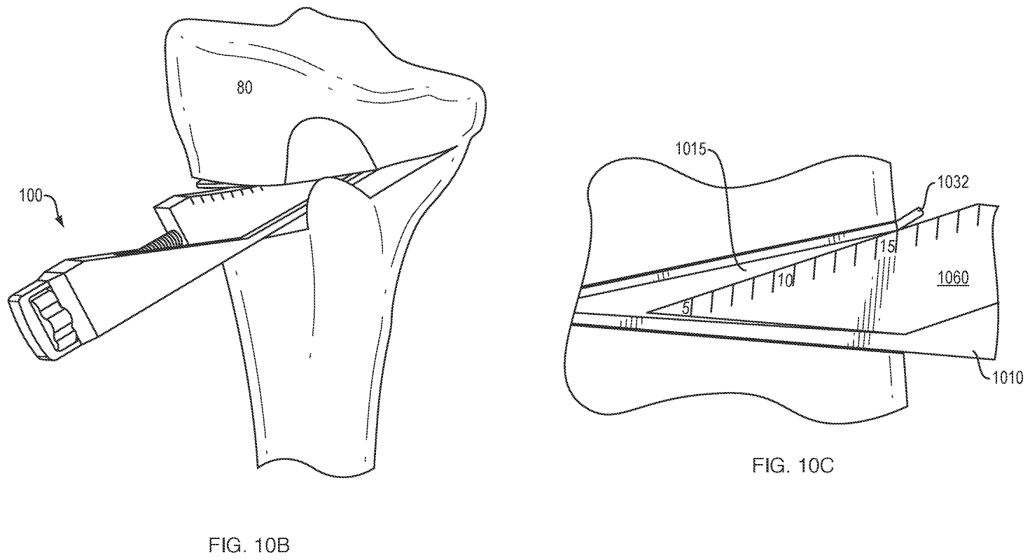

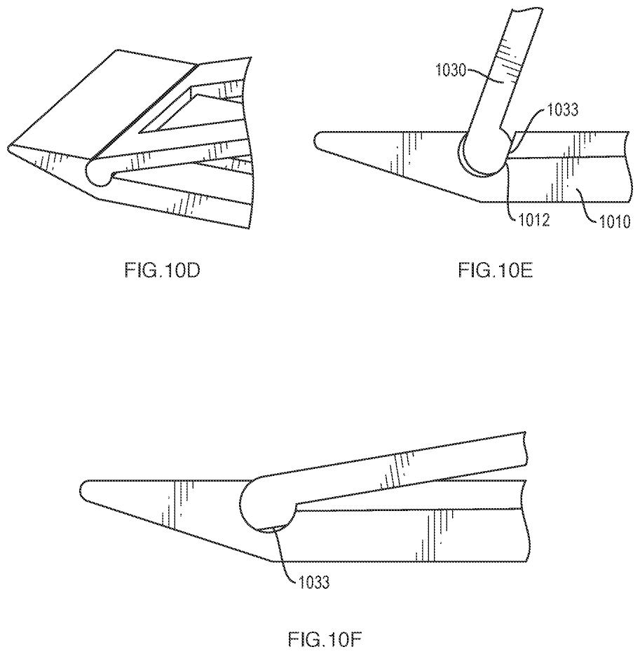

FIG. 10A illustrates a further embodiment laminar spreader that may distract the two bony segments. Laminar spreader 1000 is configured to distract the two boney segments at a controlled rate. Laminar spreader 1000 may also include a means to indicate the opening angle and control the opening to the target opening angle (γ′ or Hinge 4) that is previously determined using charts. Spreader 1000 may also include a means to maintain that opening once attained. The opening distance is planned prior to the procedure, and requires accuracy in order to improve patient outcome. The osteotomy must be opened slowly, carefully, and accurately to the appropriate opening size to mitigate cracking of the boney hinge 81. The spreader 1000 also includes a means to provide feedback as to an opening dimension. The spreader 1000 is preferably multi-use and therefore configured to be disassembled and sterilized. The spreader 1000 is preferably small, less than ½ inch wide and 4 inches long, to be placed between the two boney segments of the osteotomy while still not obscuring the target site.

Seen in FIG. 1013 shows a spreader 1000 placed between the two boney segments formed by an osteotomy of tibia 80. The instant opening angle may be 15 degrees (FIG. 10C), illustrated by numerical values on the wedge portion that align with a mark on a lifter, disclosed in more detail later. The spreader 1000 may include a base 1010 having a tip 1020 that slides into the osteotomy. Tip 1020 therefore defines a tapered end. Spreader 1000 may be hammered to initially insert tip 1020. Tip may be inserted up to the end of osteotomy initially, while in a low profile configuration. Spreader 1000 includes a lifter 1030 that may be controllably opened to engage one of the boney segments and distract the bony segments from each other. A plurality of embodiments are disclosed to control a lifter 1030. Key features of this spreader assembly 1000 include a means to control the elevation rate of the lifter 1030 at a slow rate; indication means of the opening value, or means to limit the opening value to a target value; and a hinge means that is low profile.

For example, shown in FIGS. 10A-10F is a first embodiment including a lead screw wedge mechanism. The mechanism includes a lead screw knob 1050 coupled to a lead screw 1055 that moves the wedge 1060 axially towards tip 1020 and causes the lifter 1030 to distract the osteotomy to the desired dimension. Lifter 1030 may include a free end 1032. Lifter 1030 may be pivotally coupled and may rotate relative to the base 1010, defining an adjustable cavity 1015 between the base 1010 and lifter 1030. Wedge 1060 may axially move in and out of cavity 1015 to define orientation of lifter 1030 and thereby opening dimension between the two boney segments. Wedge 1060 may engage lifter 1030 coincident with an indicator or numerical marking indicating an opening size of wedge. Shown here, the opening dimension is indicated using laser mark lines with numerical values; which correspond to osteotomy opening angles or opening gap sizes. For example the indicator on lifter 1030 lines up with the number “15” on the wedge 1060 as shown in FIGS. 10C, which may correlate to an osteotomy opening of 15 mm. Alternatively, the threaded wedge can be switched out for a patient-specific wedge that, when linearly moved forward in the assembly to a defined laser-line, for example, may open the osteotomy to the targeted opening size that the surgeon defined in pre-op. The portion of wedge 1060 disposed within cavity 1015 may be spaced away from lifter 1030. The lifter may solely engage the wedge at a discrete location coincident with the opening size indicator. Lead screw 1055 defines an axis parallel with the base longitudinal axis and may operate to move wedge linearly along the base 1010. In order to reduce the overall height of the tip 1020 of assembly 1000, the pivot defines a hardware-less and thereby low profile coupling. Illustrated in FIG. 10D-F, the lifter may be removed from the base 1010 by rotating lifter 1030 until a flat surface 1033 is parallel to the base flat surface 1012.

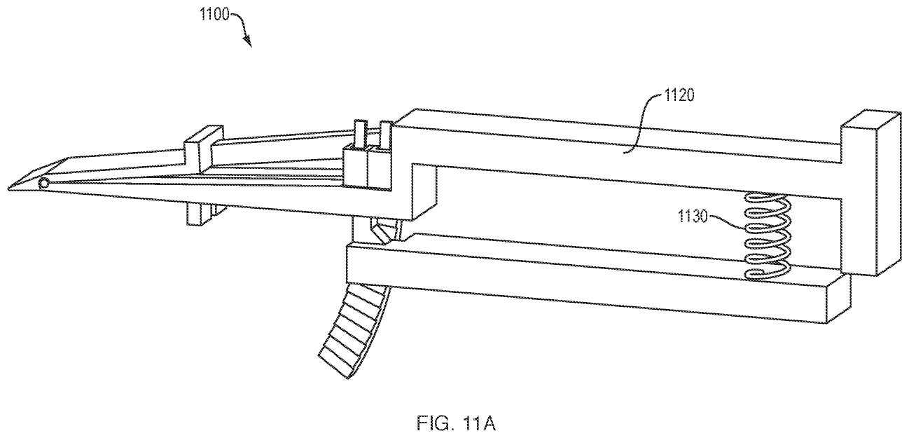

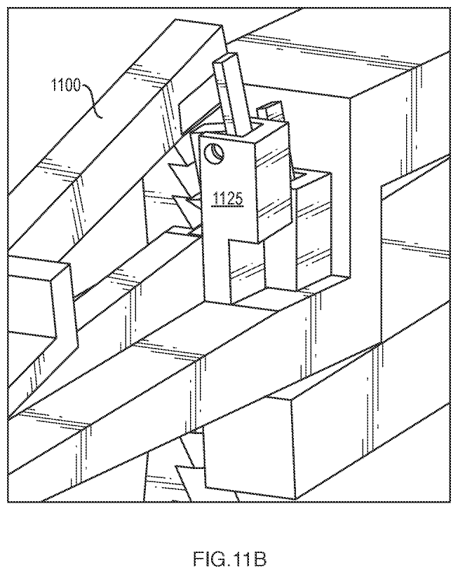

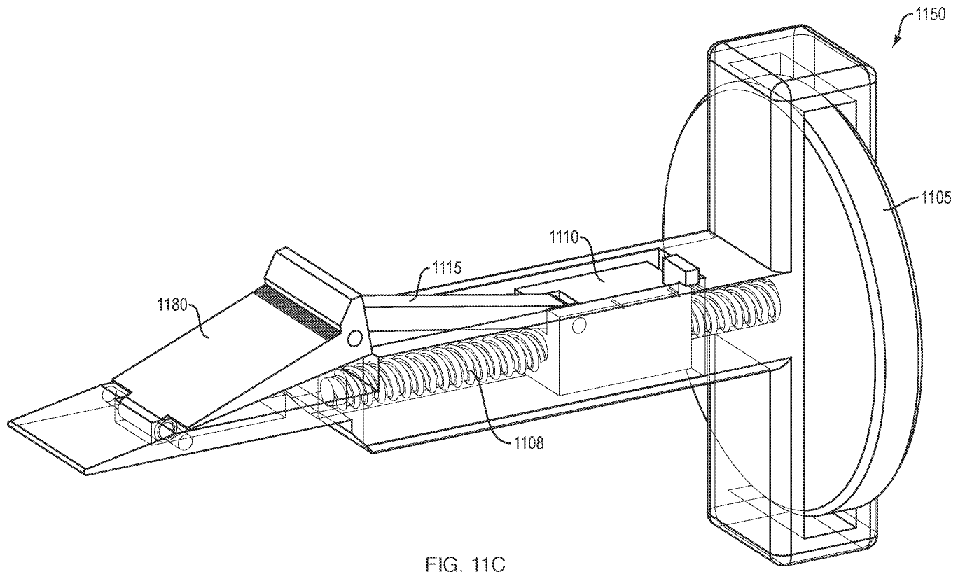

An alternate embodiment is illustrated in FIGS. 11A and 11B including a means of controlling the opening using a ratcheted lifter 1100. Actuation of a handle may rotate the ratcheted lifter 1100 to distract the osteotomy. The base 1120 may include a stationary ratchet mechanism 1125 that is operatively coupled to the lifter 1100 to rotate the lifter 1110. Each ratchet step as the lifter arm 1100 is rotated open may equate to 1 mm of wedge opening, for example, so the user may count the opening distance from audible ratchets. When it is required to be closed, tabs at the top of the ratchets can be pushed and the lifter arm is free to rotate back down into place.

FIG. 11C illustrates an alternative embodiment spreader 1150, including a linear actuator coupled to a lead screw wheel 1105 via a lead screw 1108, that, when turned, drives a linear actuator 1110 forward. This linear actuator 1110 is directly coupled to a coupling arm 1115, which is coupled to the lifter 1180; and thereby opening the osteotomy. In other alternative embodiments, a patient specific set of wedges may be provided or formed, based on the preplanning images. These wedges may include a plurality of shimmed segments that may interlock. These segments may be placed adjacent each other, to progressively increase the opening, up to a preplanned final wedge opening γ′, as determined during preplanning.

The disclosure now turns to fixation plate embodiments configured to provide fixation and optional compression to a bony hinge that may accommodate a biplanar alignment that avoids twisting of the boney hinge, as described herein. Generally, the inventors have found that modifications to the osteotomy orientation and axis of rotation leads to changes in torsional forces along the bone and torques on the bony hinge. Therefore a plurality of improved plate embodiments to account for these changes are disclosed. Firstly plate fixation may preferably be disposed directly opposite the modified hinge axis, where anatomy allows. In addition should a concomitant ACL reconstruction procedure add a tunnel to the tibia, the plate may include apertures than direct fixation members such as locking or compression screws around or away from this tunnel. The configuration of these apertures may depend on the modified hinge axis and thereby plate location. A series of plates is therefore envisioned depending on the modified hinge axis. Some plate embodiments may include contours that match anatomy of the preferred location on the bone that may be directly opposite a series of hinge axis angles, given other anatomy considerations such as the MCL. This series of plates may also include apertures specifically configured to accommodate an ACL tunnel, given the preferred location of that plate. The plate may also include an offset axis between a proximal head and distal tail of the plate, to account for torsional forces on the tibia and local anatomy considerations.

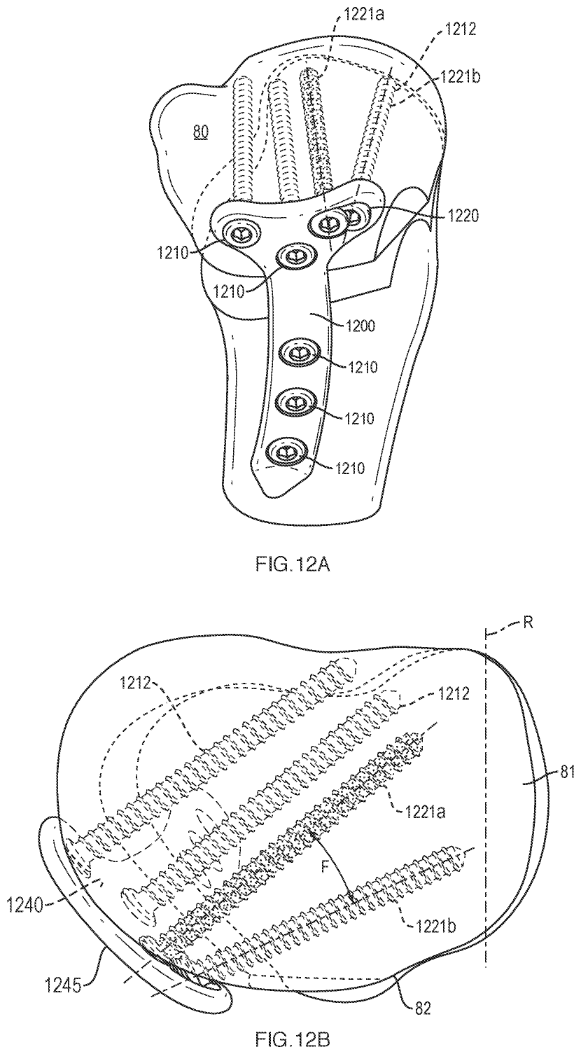

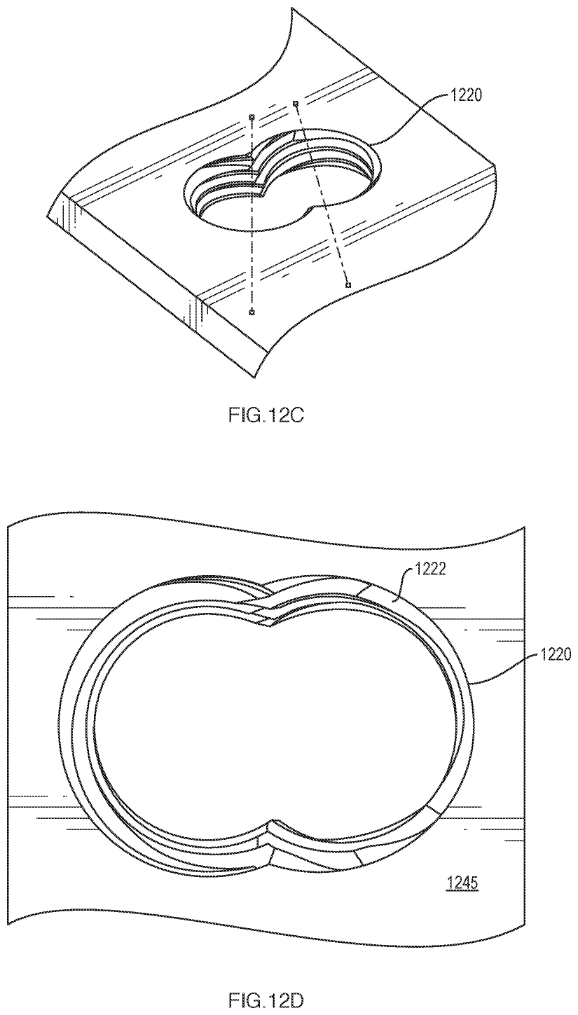

FIG. 12A shows a first example embodiment plate 1200 placed on a tibia 80 having been distracted. The plate 1200 includes a plurality of single axis holes 1210 therethough for receiving a fixation member therethrough. Plate 1200 may be “T” shaped. Holes 1210 are generally configured to receive a series of fixation members 1210 such as non-locking/locking screws for attaching plate 1200 to a portion of bone tibia 80 including over a distracted osteotomy. Plate 1200 defines a first surface 1240 shaped to engage the bone and an opposing outer surface 1245 defining a plate thickness therebetween. Plate 1200 includes at least one complex aperture 1220 having two immediately adjacent overlapping holes that extends through the plate thickness. This provides the surgeon a choice between placing a fixation member 1212 in one of two orientations. For example, a first fixation orientation 1221a allows for an orthogonal insertion where the fixation member 1212 follows that of the surrounding nearby fixation members. A second fixation orientation 1221b may be more parallel to the posterior tibial 82 surface. This second orientation 1221b may avoid a tibial ACL graft tunnel formed during combination HTO/ACL surgery or increase the distance between the nearby screws to cover a larger area in the tibial metaphysis. Therefore the two overlapping holes includes a first hole that extends along a first axis that places a fixation member in the first fixation orientation, and a second hole that extends along a second axis that places a fixation member at a second fixation orientation, that is different to the first orientation. The angle between the first and second axis may be defined at least partially given the plate location around the tibia, that may be at least defined by the modified hinge axis angle and also be configured based on a location of an ACL tunnel through the tibia. Complex aperture 1220 is configured to engage with a single fixation member only, so either orientation 1221a or 1221b may be selected, and not both, simultaneously.

Illustrated in FIGS. 12C and 12D the complex aperture 1220 extends through the plate thickness and comprises of at least two overlapping holes, having offset centers from each other. Complex aperture 1220 may include a chamfer 1222 extending from the plate surface 1245 to ease placement of a fixation member therethrough. Each of the two overlapping holes define a threaded portion to engage with threads on the fixation member 1212. The complex aperture 1220 may define a figure-of-eight shape. Complex aperture chamfer 1222 extends from a figure-of-eight shaped opening on the second (outer) surface 1245 and may intersect with the threaded portions.

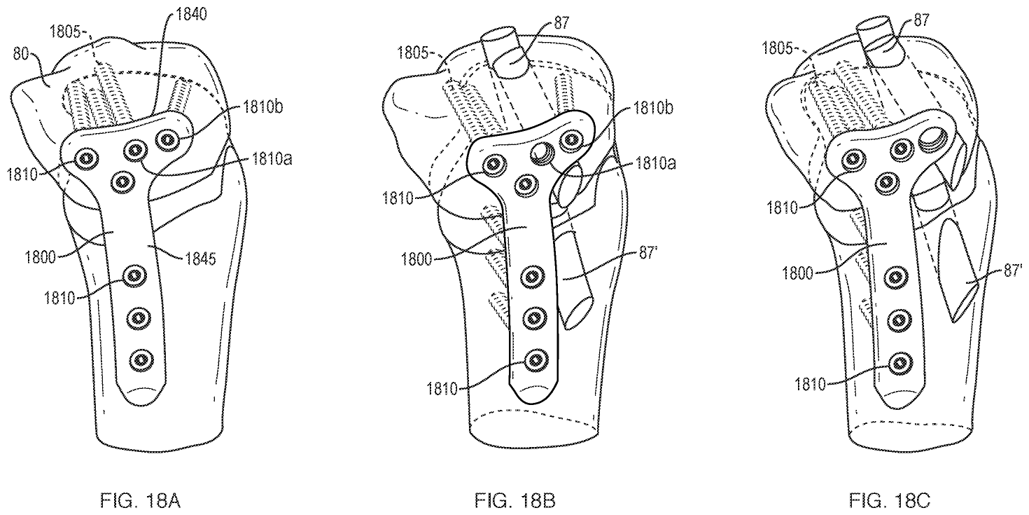

Complex aperture 1220 is preferably placed on a superior portion of plate 1200 and through a portion of the plate 1200 configured to be closest to an anterior side of the tibia. The anterior portion during ACL reconstructive surgery may have a tunnel formed through the tibia and a graft placed therealong. Depending on the tunnel location, the second axis orientation 1221b may preferably place a fixation screw in a location that will not interfere with an ACL reconstruction tunnel or meniscal root repair tunnel. FIG. 12B shows placement of a plate 1200 in a preferable location on the tibia for a hinge axis (R-R) with no rotation due to an A/P correction; rotation angle θ is zero. Plate 1200 may preferably have a contoured surface configured to generally match the shape of the tibia in that medial-anterior location as shown, reducing irritation between the adjacent soft tissues and the plate. The complex aperture 1220 may define a first axis that is orthogonal to the plate 1200 and a second angle orientated at an angle (F) relative to the first axis, between 10-30 degrees, and may preferable be about 15 degrees from the first axis. The second angle orientation preferably avoids any ACL tunnel (87 in FIGS. 18B and 18C) that may be formed through the tibia and places.

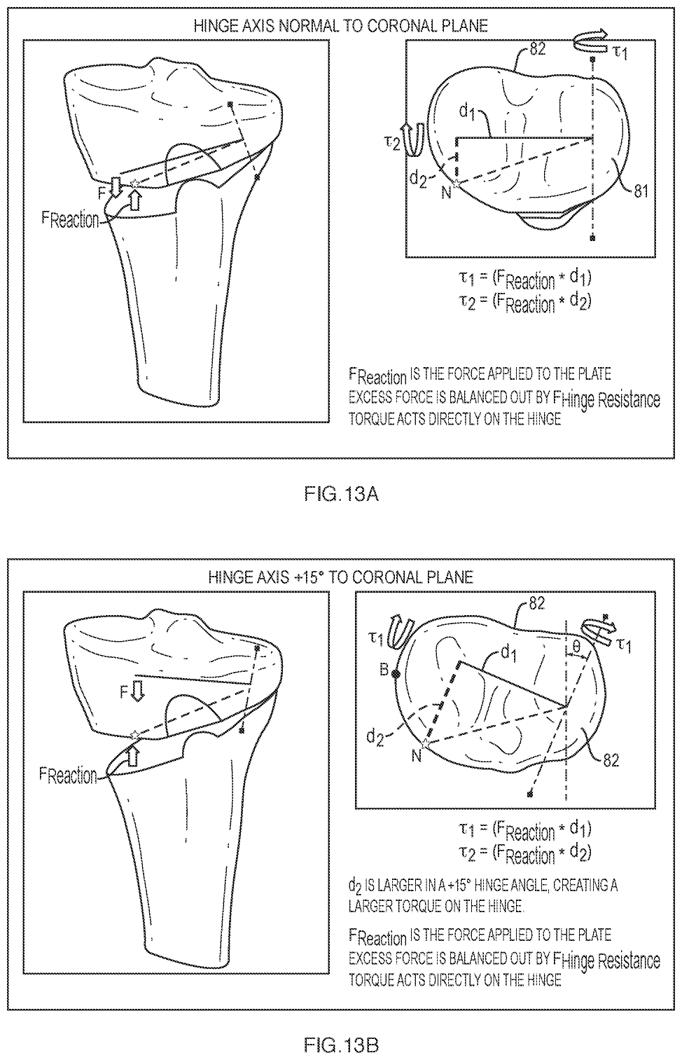

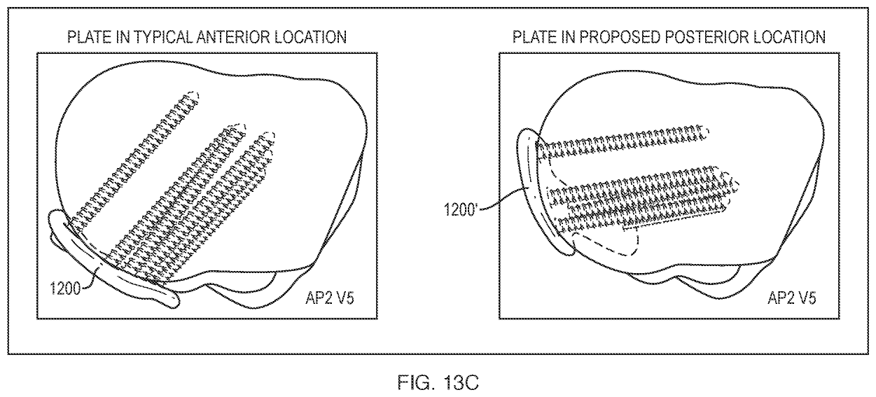

The disclosure now turns to a discussion of the changes in forces on the tibia 80 when the hinge axis angle is rotated. FIG. 13A illustrates torques τ1 and τ2 on a plate 1200 placed and centered around location N, similar to the location shown in FIG. 12B, for an unmodified plate hinge axis. Excess force is balanced out by the F_{hinge resistance}. Torque acts directly on the hinge 81. FIG. 13B represents an example case wherein a 15 degree rotation of the hinge axis may be recommended to accommodate a coronal plane correction. In this example case, the torque on the bony hinge, if the plate were centered around the same location, indicated by N, would be larger, as d₂ is larger. Therefore it would be preferable to move point N to a more medial-posterior location; for example towards point B. This reduces the dimension d₂ and thereby reduces torque on the bony hinge. Plate placement directly opposite the modified axis is not however always reasonable at the extremes, as the plate may interfere with the MCL and some neurovascular structures towards the posterior portion 82 of the tibia. The plate is preferably placed directly opposite the modified hinge axis, thereby reducing dimension d₂, limited by anatomical restrictions.

Therefore a plurality of plates may be provided, not only to accommodate the left or right patient leg, or different tibia sizes but also to accommodate an adjusted placement of a plate to reduce torque on the bony hinge 81, for a modified hinge axis angle θ. For example, for a biplanar correction for a decreased A/P correction as shown in FIG. 4C, an example plate 1200′ may preferably be contoured to be placed more medially and posteriorly relative to plate 1200, to more directly face the modified hinge axis. Plate 1200 and plate 1200′ are shown with their relative example positions in FIG. 13C. This plate 1200′ may be configured to reduce a torque on the boney hinge 81, by being preferably contoured to match a more medial outer surface of the tibia 80. Plate 1200′ may include a bi-directional complex aperture 1220′ having a first axis orientated orthogonally to the plate, and a second axis oriented at a second orientation, different than the first axis orientation. This second orientation may be between 0-30 degrees relative to the first axis. A plate 1200′ that is configured to be placed more medially and posteriorly may have a reduced angular offset between the first and second axis of these overlapping holes compared with plate 1200. For example plate 1200′ may have almost parallel axes or an axes difference of 0-5 angular degrees, while plate 1200 may have a 15 angular degree angle between the first and second axis.

A plurality of plates may be provided to accommodate a series of modified hinge axis angles. The plate may include a contour to match a preferred location based on the modified hinge axis angle. The plate may include a complex aperture as disclosed herein, the complex aperture having at least two axis orientations that may be different from each other. The two axes may be angularly offset by a value defined by the modified hinge axis angle or modified osteotomy orientation as disclosed herein. The two axes may be angularly offset to provide a plurality of placement angles of a fixation device while avoiding the ACL tunnel or MCL relative to that preferred location. For example, a first plate of the plurality of plates may include a first custom contoured shape that matches a first portion of the tibia outer surface and may also include a first complex aperture having a pair of axes angularly offset from each other to provide a means to avoid and ACL tunnel. The plurality of plates may also include a second plate including a second custom contoured shape that matches a second portion of the tibia outer surface, different than the first portion due to a modified hinge axis angle. The second plate may also include a second complex aperture having a pair of axes oriented at a second angular offset different than the first angular offset of the first plate, configured to provide a means to avoid and ACL tunnel.

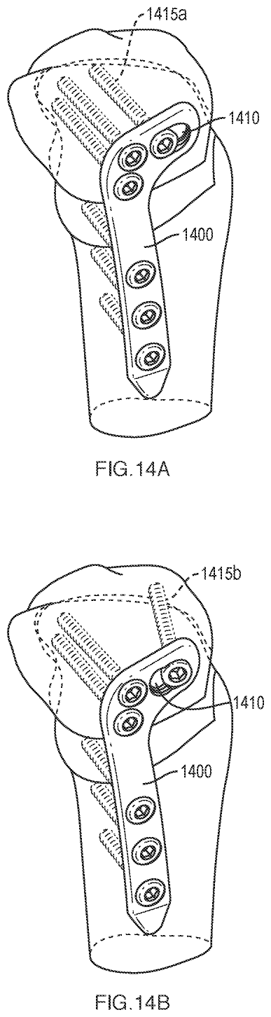

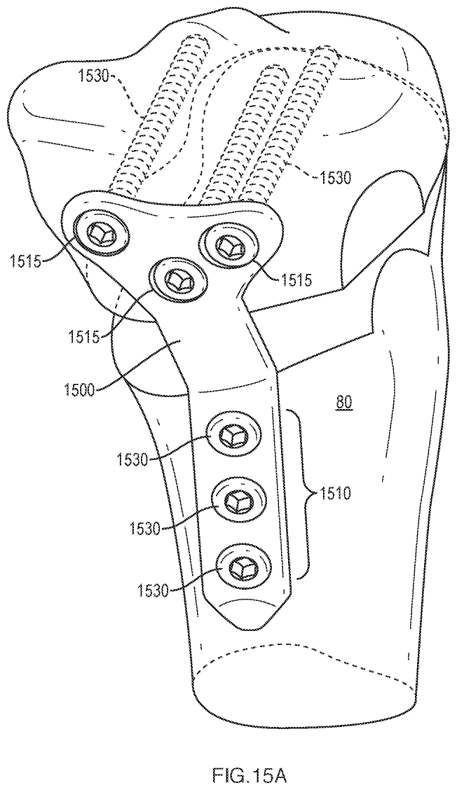

FIGS. 14A and 14B illustrate an alternative plate embodiment, configured to be placed in a more medial location around the tibia 80, similar to plate 1200′. This may accommodate the changes in torque to the boney hinge 81, as a result of rotating the hinge axis angle θ, as disclosed herein. Plate may be an “L” shape, with at least one complex aperture 1410, disposed at an end of the plate 1400 that is configured to be paced in the most anterior location of the tibia. Complex aperture 1410 is configured to receive a first fixation member 1415a or second fixation member 1415b but not both simultaneously. Complex aperture 1410 is configured to define an orientation of the first and second fixation member 1415a and 1415b, each orientation different from each other and configured to provide a choice of orientations to the surgeon, given the tibia anatomy and ACL tunnel placement, as disclosed herein.

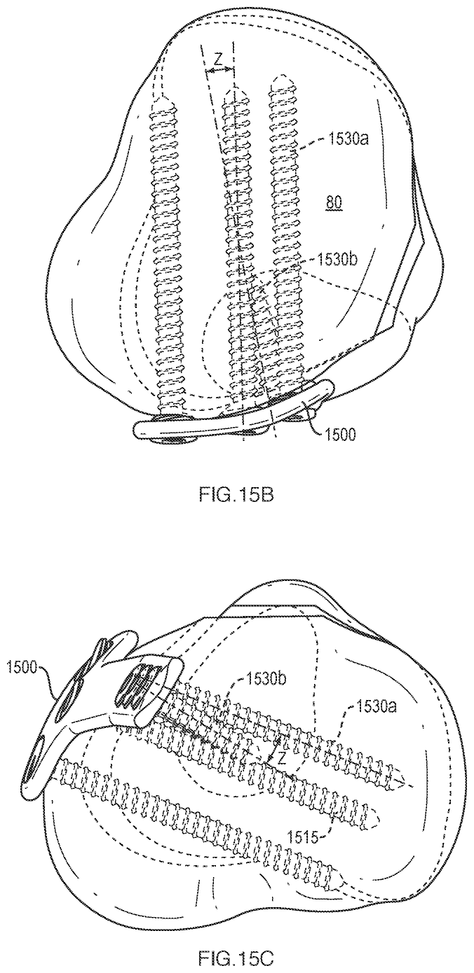

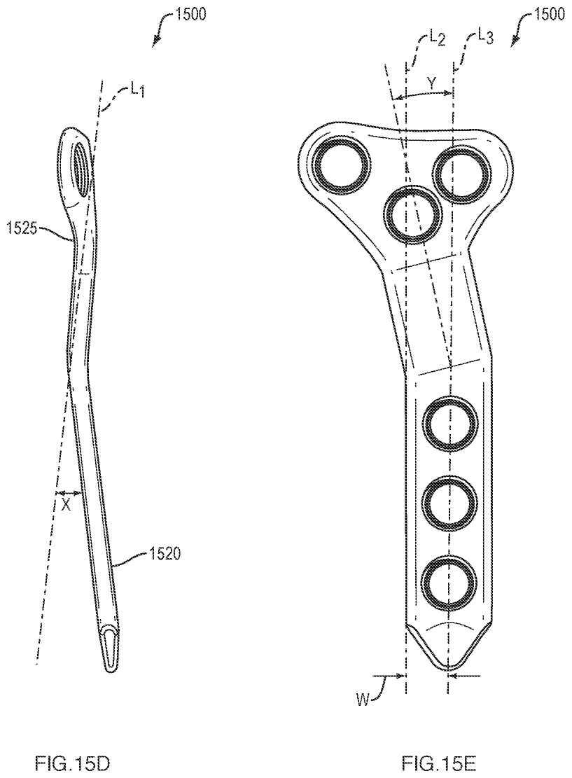

FIG. 15A-15E illustrate an alternative plate embodiment 1500, configured to be placed in a medial location around the tibia. This may accommodate the changes in torque to the bony hinge 81 as a result of rotating the hinge axis angle θ, as disclosed herein. In addition, the superior portion of this plate 1500 may be configured to be placed in a location that is spaced away from the ACL tunnel sufficiently that a complex geometry opening may not be required. This example plate 1500 includes an inferior plurality of holes 1510 and a superior plurality of holes 1515. Inferior plurality of holes 1510 may orient fixation members 1530 at a different angle to the superior plurality of holes 1515. This may account for torsional moments on the tibia 80 caused by modifying the hinge axis angle and location. FIGS. 15B and 15C illustrate the first orientation of the superior fixation members 1530a relative to the inferior most fixation members 1530b. Angle Z may increase as the plate accommodates larger modifications to the hinge axis angle. For example for an A/P correction of 4 degrees, per the chart shown in FIG. 4C, the modified angle would be approximately 14.6 degrees (correlating to the 14 degrees shown on tibia model). A plate configured for this modified angle may therefore preferably include contours matching a location around the medial portion of the tibia to reduce torque on the bony hinge, and may include a superior plurality of through-holes 1510 for orientating a plurality of fixation members at a first angle, and an inferior plurality of through-holes 1510 for orienting at least one fixation member 1530b at a second, different angle relative to the first angle. The plurality of plates may all include a superior plurality of through-holes for orientating a plurality of fixation members at a first angle, and a second inferior plurality of through-holes orienting at least one fixation member at a second, different angle relative to the first angle. Each of the plurality of plates may have a different angular offset Z between the superior plurality of through-holes and the inferior plurality, tailored for the differing torsions moments along the tibia, depending on the modified hinge axis angle value. In general the larger the modified hinge axis angle, the larger the angular offset Z.

In addition, plate inferior surface portion 1520 may be oriented at angle X to the longitudinal axis L₁ of superior portion 1525. This allows for improved apposition with the bone surfaces in this medial location. In addition, inferior portion 1520 may define a longitudinal axis L₃ extending through at least two central axes of holes 1210, L₃ offset (W) from a longitudinal axis L₂ of plate. This places the superior portion 1525 more medially on the tibia superior portion and more anteriorly on the tibia inferior side of the osteotomy, to center the inferior stem 1520 along the tibia and resist torsional moments on the tibia as a result of the modified hinge axis angle. A plate that accommodate a larger modified hinge axis angle, may have larger values of W and X relative to a plate that accommodates a smaller modified hinge axis angle.

The disclosure now turns to a system 1600 for adjusting and increasing compression to a lateral bony hinge of an HTO. This system may be applicable for both the more traditional single plane open-wedge high tibial osteotomy as well as the biplanar HTO as disclosed herein. Increased compression on the bony hinge 81 may improve the patient outcome in a variety of ways. It may facilitate faster bone healing during an HTO procedure; it may decrease a chance of non-union should the bony lateral hinge crack during the procedure; and it may allow for earlier weight bearing on the osteotomy as it preloads the osteotomy, which decreases the chance of a loss of correction during the healing process. Generally this system includes a means of engaging a fixation plate, such as fixation plate 1200 for example and a means of adjustably elastically bending or flexing the plate 1200. Once a portion of the plate in this flexed state is fixed to the bone, the bony hinge 81 may be fixed in this compressed state.

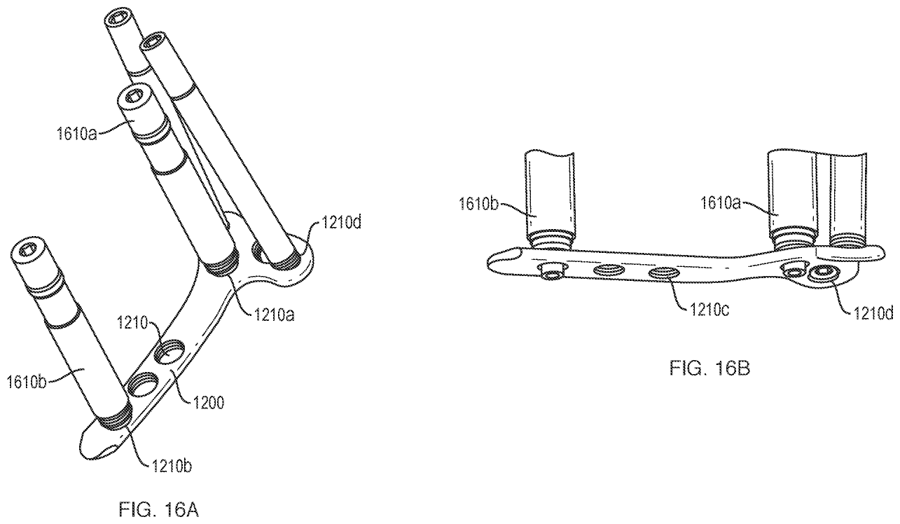

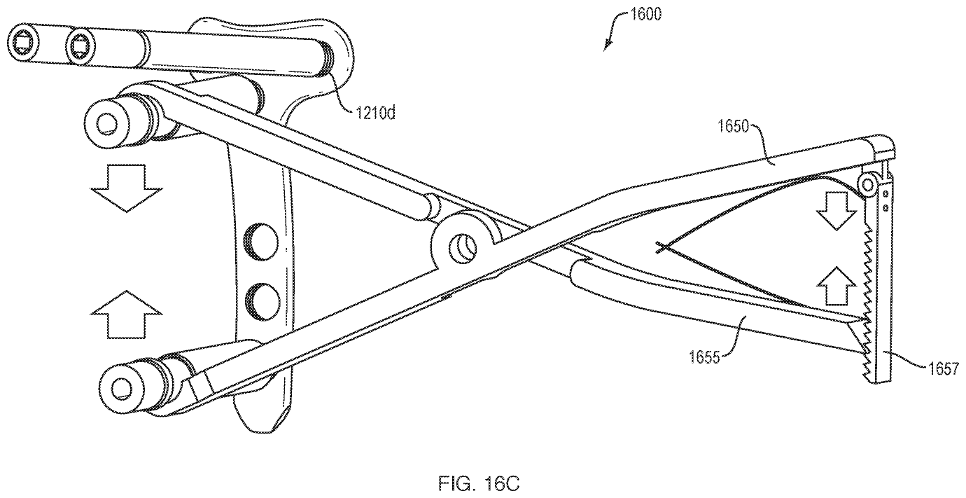

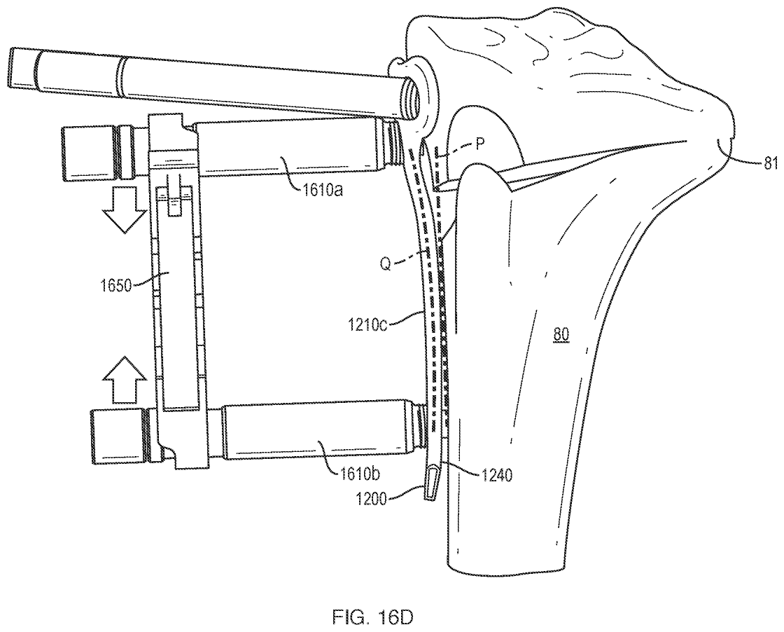

FIG. 16A shows an example fixation plate, that may be similar to plate 1200 disclosed herein with a plurality of holes 1210 therethough. Holes 1210 are generally configured to receive a series of fixation means therethough, such as non-locking/locking screws, for attaching plate 1200 to a portion of bone 80 including over a distracted portion of bone. Holes 1210 may be threaded. Plate 1200 is an exemplary shape and the number of holes 1210 and location may differ. System 1600 includes a means of engaging the plate 1200 at spaced apart locations, preferably at two locations, one towards a superior end and one towards an inferior end of the plate 1200. In this embodiment, a first shaft 1610a may threadingly couple to hole 1210a located at a superior end of plate. First shaft 1610a may be a first drill guide 1610a. Hole 1210a is disposed on a superior side of the osteotomy, and may be disposed between the osteotomy and other more superiorly located fixation holes 1210d. A second shaft that may be a second drill guide 1610b may threadingly engage a hole 1210b through the plate 1200, hole 1210b being disposed towards an inferior end of the plate 1200, on an inferior side of osteotomy. Hole 1210b may be the inferior-most hole on the plate 1200. There is preferably at least one fixation hole, such as holes 1210c between hole 1210a and 1210b, so that a fixation means may extend through hole 1210c and fix the plate 1200 while in the flexed or stressed configuration. It is preferable that flexing is primarily along a longitudinal axis of the plate. Drill guides 1610a, 1610b may both allow a drill tip therethrough and therefore may be cannulated to direct an orientation of a drill tip into the bone 80. Drill guides 1610a and 1610b may include tips that threadingly engage a corresponding threaded hole 1210. These threaded tips may extend through and beyond the associated hole 1210, providing a standoff between a bone facing surface 1240 of the plate 1200 and the bone 80. A standoff distance may be between 1 mm and 5 mm. FIG. 16B illustrates the drill guides 1610a and 1610b extending through and beyond the fixation holes 1210. With only the drill guides forming standoff of up to 5 mm, some limited compression may be achieved while fixing the plate though hole 1210c. However, additional compression on the bone may be beneficial.

Turning now to FIG. 16C, system 1600 also includes a tool 1650 that imparts additional flex to the plate 1200. An external compression tool 1650 may engage the shafts 1610a and 1610b and flex the plate 1200. External compression tool 1650 may be disposed externally to the patient during use, with the two shafts 1610a and 1610b extending through skin incisions. Generally, compression tool 1650 is configured to engage the two shafts 1610a and 1610b and move the axis of each shaft so as to flex the plate 1200. There are a plurality of ways of achieving this. For example, external compression tool 1650 may be a pivoting clamp or pliers style tool that extends transversely away from longitudinal axes of the shafts 1610. Tool 1650 engages drill guides 1610a and 1610b, and rotates the longitudinal axis of each shaft to impart stress on the plate and elastically flex plate 1200. In this example system 1600, tool distal end 1652 at least partially encircles each drill guide 1601a and 1610b. Squeezing handle end 1655 rotates the two drill guides longitudinal axes relative to each other and imparts a longitudinal flexing on plate 1200. Handle end 1655 may include a means to maintain the flexed state. Ratcheting means 1657 are illustrated. Each successive ratchet tooth increases the stress on the plate 1200 and thereby compression on the bony hinge 81. In other embodiments, a threaded member may be operatively coupled to and between the handle end 1655, such that actuation of the threaded member may adjust the distance between the handles, to move the shaft longitudinal axes relative to each other and imparts a longitudinal flexing on plate 1200. In some embodiments, the longitudinal axes of each shaft 1610 may rotate from being approximately parallel with each other to non-parallel. Other example embodiments of a tool 1650 may be inserted into each of the cannulated end of the drill guide 1610. Tool 1650 may extend longitudinally from shafts 1610a and 1610b. Tool 1650 may include a ratchet mechanism that extends directly between the two shafts 1610a and 1610b, and the user may squeeze the two shaft handle ends towards each other to engage a successive set of teeth on the ratchet. Alternatively a threaded member may extend between the two shafts 1610a and 1610b, and actuating the threaded member may alter the distance between the two shafts 1610a and 1610b. FIG. 16D illustrates a plate 1200 on bone 80, with the system 1600 engaged and elastically flexing the plate 1200. Plate 1200 may align with line pin the relaxed state, and upon flexing the plate via tool 1650 and via drill guides 1610a and 1610b, plate 1200 may elastically flex to align with line Q. Placing fixation means through holes 1210c while the plate is flexed in this shape imparts higher compression on the bony hinge 81 than with the plate less flexed or in a relaxed state.

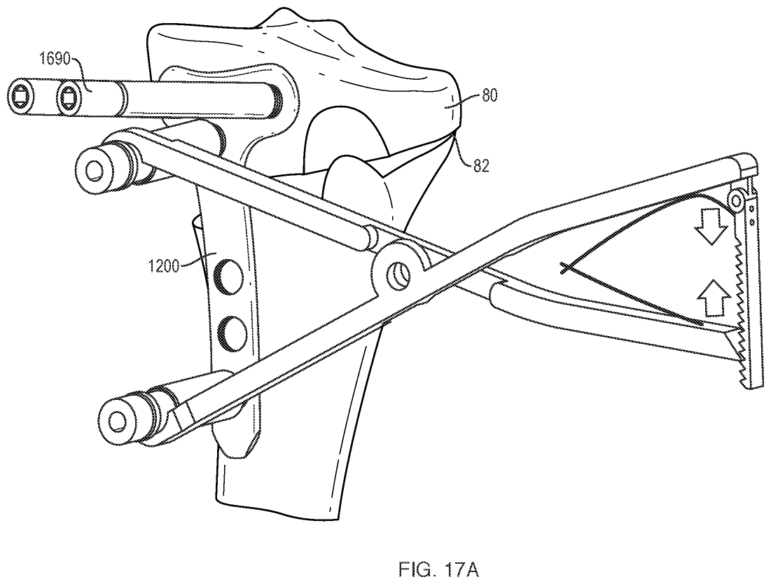

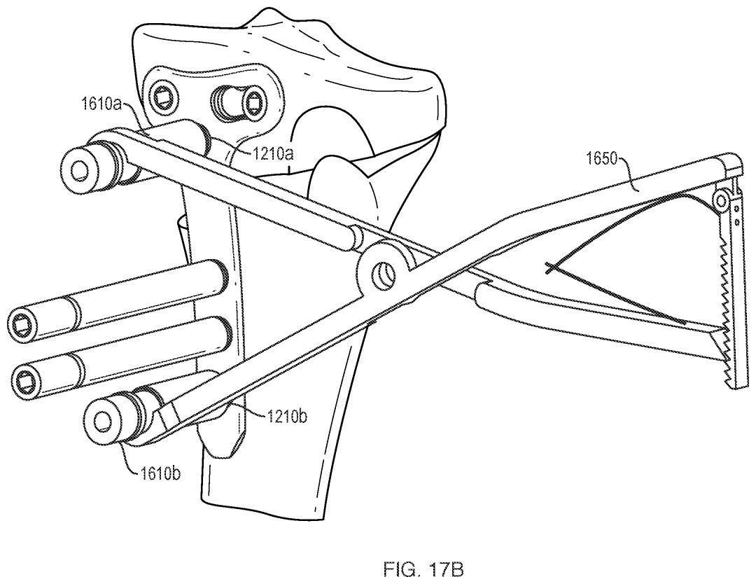

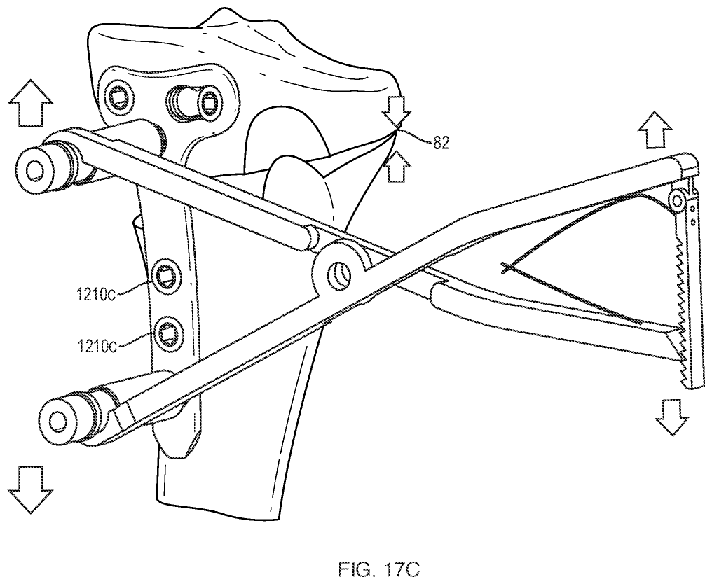

A method of adjustably compressing a bony hinge 81 therefore is illustrated in FIG. 17A-17C. Fixation plate 1200 may be placed on the target bone 80, either side of the distraction. A superior portion of the plate may be fixed with the bone 80 using guides 1690 and locking screws. Shafts, which may be drill guides 1610a and 1610b may be coupled to plate 1200 and may extend through and beyond the bone-facing surface 1240 of the plate 1200 to space the plate 1200 away from the bone outer surface. Drill guides each define a longitudinal axis and have a length defining a handle end, such that each drill guide 1610 may be engaged with the plate by hand, and at least one of the drill guides may be used to handle and place the plate 1200 on the bone 80. A superior drill guide 1610a may extend through an incision formed in the patent to place the plate. A second minimal incision may be formed more inferiorly to allow access for the second drill guide 1610b to the plate 1200. Two discrete connecting locations between the drill guide 1610 and plate 1200 may be preferable to reduce incision sizes through the patient skin. First drill guide 1610a may be coupled through a hole 1210a that is disposed between the superior-most holes and the distracted bone. Second drill guide 1610b may be coupled to a hole 1210b that is an inferior-most hole of the plate 1200. External compression tool 1650 may then engage the drill guides 1610a and 1610b and operated to elastically flex the plate 1200 before fixation means are placed through holes 1210c, so as to compress lateral hinge 82 of bone 80. Tool 1650 may have a series of settings to adjustably flex the plate 1200 and thereby adjustable compression on the hinge 81. After fixation via holes 1210c, tool 1650 may then be released (FIG. 17C). Drill guides 1610a and 1610b may then be used to guide a drill along a targeted trajectory. Drill guides 1610a and 1610b may then be removed and plate 1200 further fixed to the bone via holes 1210 and 1210 (not shown). Stressing the plate 1200 can be carefully planned and made precise and accurate to what is needed clinically.

FIG. 18A shows a further example embodiment plate 1800 placed on a tibia having been distracted, with a plurality of single axis holes 1810 therethough for receiving a fixation member therethrough. Plate 1800 may be an asymmetrical “T” shape. Holes 1810 are generally configured to receive a series of fixation members 1805 therethrough such as non-locking/locking screws for attaching plate 1800 to a portion of bone tibia 80 that may include over a distracted osteotomy. Plate 1800 defines a first surface 1840 shaped to engage the bone and an opposing outer surface 1845 defining a plate thickness therebetween. Unlike plate 1200, plate 1800 may not include a complex aperture 1220, but instead may include two separate holes 1810a and 1810b, that are directly adjacent each other on a superior end of plate 1800. The two holes 1810a and 1810b may have axes parallel to each other or, as shown may be angularly offset from each other. In this example embodiment, hole 1810a may have an axis that is orthogonal to the plate 1800 and may be parallel to other holes directly adjacent to it. Hole 1810b may define a central axis that is angularly offset by about 15 angular degrees)(° from a central axis of hole 1810a. These two holes 1810a, 1810b are configured to provide at least one hole, 1810a or 1810b that allows a fixation member 1205 a path through the tibia that avoids an ACL tunnel 87 or 87′. However, unlike a complex aperture, fixation members 1205 may also extend through both holes (1810a, 1810b) simultaneously, should the anatomy allow, hence increasing plate fixation overall. FIG. 18B shows a plate 1800 placed in a more anterior position on the tibia 80, wherein a fixation member 1205 is preferably placed through hole 1810b and not hole 1810a, to avoid the ACL tunnel 87 or 87′. Explained earlier, a fixation plate is preferably placed directly opposite a hinge axis, for torque considerations, and therefore a plate may be placed more anteriorly for a hinge axis rotation in a direction that increases the tibial plateau slope, as shown in FIG. 4D. No fixation member 1205 is placed though hole 1810a. Alternatively, FIG. 18C shows plate 1800 placed in a more medial position on the tibia 80, wherein a fixation member 1205 is preferably placed through hole 1810a and not hole 1810d, to avoid the ACL tunnel 87 or 87′. This may correlate to a biplanar correction that decreases the tibial plateau slop, as shown in FIG. 4C. No fixation member 1205 is placed though hole 1810b. Turning back to FIG. 18A, depending on the concomitant surgeries and location of the ACL, both holes 1810a and 1810b may receiving a fixation member 1205 therethrough, to increase fixation with the tibia 80. Therefore the two holes (1810a, 1810b) includes a first hole that extends along a first axis that places a fixation member in the first fixation orientation, and a second separate hole that extends along a second axis that places a fixation member 1205 at a second fixation orientation, that is different to the first orientation. The angle between the first and second axis may be defined at least partially given the plate location around the tibia, that may be at least defined by the modified hinge axis angle and also be configured based on a location of an ACL tunnel through the tibia.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A surgical method of attachment of a first bony segment in relation with a second bony segment, both segments belonging to a same bone, which comprises the steps of:
cutting said bone partially, to separate it in two bony segments linked together by a bony hinge; and
rotating both bony segments around said bony hinge, wherein the bony hinge is configured to rotate both bony segments around a single degree of freedom of the bony hinge to a final alignment that accommodates a correction in two substantially orthogonal correction planes.

2. The method of claim 1 further comprising the preoperative steps of:
determining a relative three-dimensional positions of bony segments to obtain the final alignment of the two bony segments, the relative three-dimensional position including a rotation about the two substantially orthogonal correction planes; and
based on the relative three dimensional positions of both bony segment, determining an orientation of the bony hinge.

3. The method of claim 1 wherein the two substantially orthogonal planes include the sagittal plane and the coronal plane.

4. The method of claim 2 wherein determining the orientation of the bony hinge, comprises using either a look-up table, computer program or mechanical computer.

5. The method of claim 2 wherein after determining the orientation of the bony hinge, drilling a hole through the bone along the determined orientation.

6. The method of claim 2 wherein determining the orientation of the bony hinge comprises determining a rotation angle of a hinge axis about a longitudinal axis of the bone and relative to a plane through the bone, the plane parallel to the either the sagittal plane or coronal plane.

7. The method of claim 2 wherein determining the orientation of the bony hinge comprises determining a rotation angle of a hinge axis about a longitudinal axis of the bone and relative to a plane through the bone and also determining a final wedge opening angle between the two boney segments.

8. The method of claim 7 wherein determining the final wedge opening angle comprises either using a look-up table, mechanical computer or a computer program.

9. The method of claim 1 further comprising fixing the first bony segment in relation with the second bony segment in the final alignment using a fixation plate, the fixation plate configured to accommodate induced loads on the bone as a result of rotating both bony segments around the single degree of freedom of the bony hinge to the final alignment that accommodates the correction in the two substantially orthogonal correction planes.

10. A surgical method of attachment of a first bony segment in relation with a second bony segment accommodating a biplanar correction, including a first and a second plane perpendicular to the first plane, and wherein both segments belong to a same bone, comprising:
cutting partially into the bone, cutting being implemented until obtaining a partial cut which separates partially said bone in two bony segments linked together by a bony hinge, the bony hinge defining a hinge axis that is oriented at a non-zero angle relative to both the first and second plane through the bone;
rotating said first bony segment with respect to said second bony segment around said bony hinge about a single degree of freedom until obtaining a desired three-dimensional alignment of the two bony segments, including the biplanar correction and reaching a position in which facing sides of said bony segments are separated from each other by a predetermined angle.

11. The method of claim 10 further comprising predetermining a hinge axis angle about a longitudinal axis of the bone and relative to the first plane using both a varus correction angle input and an anterior-posterior slope correction angle input and cutting partially into the bone includes drilling a hole along the predetermined hinge axis angle.

12. The method of claim 11 wherein predetermining the hinge axis angle comprises using physical charts, mechanical computer or a computer program.

13. The method of claim 11 wherein drilling the hole includes using preplanning navigation.

14. The method of claim 11 wherein drilling the hole includes using a computer piloted robot whose working head is able to be moved according to at least three degrees of freedom.

15. The method of claim 10 wherein cutting is implemented by using a computer piloted robot whose working head is configured to move according to at least three degrees of freedom, the working head supporting a cutting tool.

16. The method of claim 10 wherein cutting is implemented using a cutting guide.

17. A surgical method of attachment of a first bony segment in relation with a second bony segment, both segments belonging to a same bone, which comprises the steps of:
determining a modified hinge axis angle of a boney hinge that provides a single degree of freedom rotation of the first bony segment relative to the second bony segment, the hinge axis angle accounting for a final desired alignment of the two bony segments including two substantially orthogonal plane corrections;
placing an aimer assembly around the bone and orienting a guide tube of the aimer assembly in a first orientation;
moving the guide tube along an aimer arm of the aimer assembly to orient guide tube along the modified hinge axis angle; and
inserting a drill through the guide tube and forming a hole through the bone defining the hinge axis angle of the bony hinge.

18. The method of claim 17 wherein moving the guide tube includes sliding the guide tube along the aimer arm.

19. The method of claim 17 wherein the first orientation is defined as an orientation accounting for only one of the two substantially orthogonal plane corrections.

20. The method of claim 17 wherein the guide tube is configures to receive a hinge pin.
